(19) United States
(12) Patent Application Publication
(10) Pub. No.: US 2009/0286721 A1
(43) Pub. Date: Nov. 19, 2009

(54) TARGETED PLASMINOGEN ACTIVATOR FUSION PROTEINS AS THROMOBOLYTIC AGENTS

(76) Inventors: Junliang Pan, El Cerrito, CA (US); Qingyu Wu, Lafayette, CA (US); Achim Schuttler, Aachen (DE); Annemarie Schuttler, Aachen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/817,454

(22) PCT Filed: Sep. 6, 2005
(86) PCT No.: PCT/EP2005/009553

§ 371 Date: May 14, 2009

(30) Foreign Application Priority Data
Mar. 4, 2005 (JP) 2005-61685

(51) Int. Cl. A61K 38/00; C07K 19/00; C07K 16/00; A61P 7/00; A61P 9/00; C07H 21/04; A61K 48/00

(52) U.S. Cl. 514/12; 530/350; 530/391.1; 536/23.4; 514/44 R

(57) ABSTRACT

This invention relates to novel fusion proteins, comprising a targeting protein and a plasminogen activator, preferably an antibody that binds to P-selectin, operably linked to the plasminogen activator DSPAalpha1, or analogs, fragments, derivatives, or variants thereof, which are useful as thrombolytic agents. Pharmaceutical compositions containing these fusion proteins, methods of using these fusion proteins as thrombolytic agents, and processes for synthesizing these fusion proteins are also described herein.

### FIELD OF THE INVENTION

The invention relates to novel fusion proteins, useful as thrombolytic agents, comprising a targeting protein operably linked to a fibrin-selective plasminogen activator, or analogs, fragments, derivatives, or variants thereof. In a preferred embodiment the targeting protein binds to the surface of activated platelets or activated endothelial cells.

### BACKGROUND OF THE INVENTION

Arterial thrombosis, which is a life-threatening disease that affects millions of humans each year, is caused by uncontrolled coagulation and platelet aggregation within a damaged blood vessel. Excessive fibrin and platelet clots formed in the vessel block blood flow, causing ischemic damage to vital tissues or organs. Approved therapeutic approaches to treat arterial thrombosis, such as myocardial infarction, use plasminogen activators in combination with antiplatelet drugs and anticoagulants. Plasminogen activators currently used include tissue-type plasminogen activator (“tPA”), urokinase (“uPA”), and streptokinase. Administration of these thrombolytic agents in the setting of occlusive thrombus enhances the rate of fibrin degradation, restoring arterial patency and blood flow to ischemic tissues. Coronary thrombolytic therapy has reduced mortality in patients with acute myocardial infarction (see Collen, D. and Lijnen, H. R., Blood (1991), Vol. 78, pp. 3114-3124; Topol, E. J., Prog. Cardiovasc. Dis. (1991), Vol. 34, pp. 165-178); Verstraete, M. et al., Drugs (1995), Vol. 50, pp. 29-42; Verstraete, M. and Zoldhelyi, P., Drugs (1995), Vol. 49, pp. 856-884; and Huber, K. and Maurer, G., Semin. Thromb. Hemost (1996), Vol. 22, pp. 15-26). Major limitations of current thrombolytic therapy include bleeding, most notably intracranial hemorrhage, failure to achieve adequate myocardial reperfusion, and coronary reocclusion. Since the introduction of current dosing regimens (e.g., front loaded tPA), however, little progress has been made to further improve the rate and extent of coronary thrombolysis, or to reduce the bleeding risk. As such, newer agents are needed.

Plasminogen activators with improved thrombolytic properties have been developed. TNK-tPA is a variant of tPA generated by recombinant DNA technology. TNK-tPA is more resistant to inhibition by plasminogen activator inhibitor-1 (“PAI-1”), has a longer half-life in the circulation, and can be administered as a single bolus injection (Collen, D. et al., Thromb. Haemost. (1994), Vol. 72, pp. 98-104; and Keyt, B. A. et al., Proc. Natl. Acad. Sci. USA (1994), Vol. 91, pp. 3670-3674). Reteplase is an unglycosylated protein consisting of only the kringle 2 and protease domains of tPA. Reteplase has a longer plasma half-life, but is less fibrin-specific than tPA (Martin, U. et al., Thromb. Haemost (1991), Vol. 66, pp. 569-574). Currently, both TNK-tPA and Reteplase have been approved for clinical use.

One of the major limitations of current thrombolytic therapy is bleeding risk (Rao, A. K. et al., J. Am. Coll. Cardiol. (1988), Vol. 11, pp. 1-11; and Arnold, A. E. et al., J. Am. Coll. Cardiol. (1989), Vol. 14, pp. 581-588). Approximately 5-10% of patients treated with thrombolytic therapy experience bleeding episodes. Among these bleeding episodes, close to 10% were intracranial hemorrhage, which can be fatal (see Chesebro, J. H. et al., Cardiol. Clin. (1988), Vol. 6, pp. 119-137; Topol, E. J. et al., J. Am. Coli. Cardiol. (1987a), Vol. 9, pp. 1205-1213; Topol, E. J. et al., J. Am. Coll. Cardiol. (1987b), Vol. 9, pp. 1214-1218; PRIMI Trial Study Group, Lancet (1989), Vol. 1, pp. 863-868; and ISIS Collaborative Group, Lancet (1992), Vol. 339, pp. 753-770). The bleeding is due mainly to nonspecific cleavage of fibrinogen and excessive proteolysis of an aged (old) hemostatic fibrin clot by the exogenous plasminogen activator. In addition to bleeding, thrombolytic agents have other limitations. Up to 25% of patients with acute myocardial infarction are resistant to current thrombolytic regimens. In these patients, there is no significant myocardial reperfusion to the ischemic heart. In another 30-40% of patients, therapy only achieves partial reperfusion within 90 minutes, a critical time window to minimize cell death in the ischemic-tissue (GUSTO Angiographic Investigators, N. Engl. J. Med. (1993), Vol. 329, pp. 1615-1622; and Barbagelata, N. A. et al., Am. Heart J. (1997), Vol. 133, pp. 273-282). Furthermore, approximately 30% of patients experience acute coronary reocclusion following thrombolytic therapy. The ongoing activation of platelets in occlusive thrombus is believed to contribute greatly to failure of thrombolytic therapy (Fay, W. P. et al., Blood (1994), Vol. 83, pp. 351-356; Stringer, H. A. et al., Arterioscler. Thromb. (1994), Vol. 14, pp. 1452-1458; Torr-Brown, S. R. and Sobel, B. E., Thromb. Res. (˜1993), Vol. 72, pp. 413-421; Jang, I. K. et al., Circulation (1989), Vol. 79, pp. 920-928; and Kunitada, S. et al., Blood (1992), Vol. 79, pp. 1420-1427). In both in vitro and in vivo studies, platelet-rich clots were found to be more resistant to thrombolysis than fibrin-rich, platelet-poor clots (Bode, C. et al., Circulation (1991), Vol. 84, pp. 805-813; and Coller, B. S., N. Engl. J. Med. (1990), Vol. 322, pp. 33-42).

P-selectin is a ˜140 kDa glycoprotein that is stored mainly in the alpha-granule of resting platelets. Upon platelet activation, P-selectin is rapidly translocated to the cell surface where it facilitates platelet and leukocyte interactions in the damaged vessel wall. The cell surface expression of P-selectin on activated platelets lasts no more than a few hours (McEver, R. P., Thromb. Haemost (1991), Vol. 65, pp. 223-228; McEver, R. P., Curr. Opin. Immunol. (1994), Vol. 6, pp. 75-84; and Lasky, L. A., Science (1992), Vol. 258, pp. 964-969). In addition to platelets, P-selectin is also present in the Weibel-Palade body of normal endothelial cells. Upon the activation of endothelial cells by histamine and thrombin, P-selectin is rapidly re-distributed to the cell surface. Activated platelets are major cellular components within a freshly formed thrombus, and activated endothelial cells are abundant near the site of vascular damage. In contrast, the major components in an aged (old) hemostatic plug are fibrin molecules.

Four tPA-like proteins were derived from the saliva of the vampire bat Desmodus rotundus: DSPAalpha1, DSPAalpha2, DSPAbeta, and DSPAgamma (Gardell, S. J. et al., J. Biol. Chem. (1989), Vol. 264, pp. 17947-17952; and Kratzschmar, J. et al., Gene (1991), Vol. 105, pp. 229-237). Of these, DSPAalpha1 is the longest protein and, structurally, most similar to human tPA. Unlike other plasminogen activators that cleave both fibrinogen and fibrin, DSPAalpha1 is highly specific for fibrin (Bringmann, P. et al. (1995), Vol. 270, pp. 25596-25603). DSPAalpha1 has several hundred-fold greater fibrin-specificity than tPA (Bringmann, P. et al. (1995), supra; see Toschi, L. et al., Eur. J. Biochem. (1998), Vol. 252, pp. 108-112; Stewart, R. J. et al., J. Biol. Chem. (1998), Vol. 273, pp. 18292-18299; and Schleuning, W. D. et al., Ann. N.Y. Acad. Sci. (1992), Vol. 667, pp. 395-403. In animal models of thrombolysis, DSPAalpha1 is more potent than tPA (Gardell, S. J. et al., Circulation (1991), Vol. 84, pp. 244-253; Witt, W. et al., Blood (1992), Vol. 79, pp. 1213-1217; and Witt, W. et al., Circulation (1994), Vol. 90, pp. 421-426). DSPAalpha1 (Desmoteplase) is in clinical development for the treatment of stroke.

U.S. Pat. No. 6,008,019 discloses the four DSPA proteins, and claims the use of DSPAalpha1 as a thrombolytic agent. U.S. Pat. No. 5,830,849 discloses and claims the use of DSPAalpha2 as a thrombolytic agent.

Structurally, DSPAalpha1 and DSPAalpha2 consist of four distinct domains: a fibronectin-like finger (“finger”) domain, an epidermal growth factor (“EGF”) domain, a kringle domain, and a serine protease domain; DSPAbeta consists of three distinct domains: an EGF domain, a kringle domain, and a serine protease domain; and DSPAgamma consists of two distinct domains: a kringle domain and a serine protease domain (Kratzschmar, J. et al. (1991), supra).

Structure-function analysis demonstrates that the finger domain contributes to the fibrin dependence and selectivity of DSPAalpha1 (Bringmann, P. et al. (1995), supra). The increased fibrin specificity of DSPAalpha1 as compared to tPA appears to be due to the fact that DSPAalpha1 has one kringle domain, whereas tPA has two kringle domains (Stewart, R. J. et al. (1998), supra). Other structure-function analysis suggest distinct modifications of the native t-PA amino acid sequence—e.g. in the cymogene triade—in order to increase t-PA's fibrin specificity (EP 1 308 166 A1).

### SUMMARY OF THE INVENTION

The present invention provides novel fusion proteins, which act as thrombolytic agents, comprising a targeting protein, operably linked to a plasminogen, activator, or analogs, fragments, derivatives, or variants thereof, wherein the targeting protein binds to a vascular damage related biological structure. A “vascular damage related biological structure” according to the invention is any biological molecule or structure which is indicative for vascular damage either in terms of quantity or quality. Examples for vascular damage related biological structures are surface molecules on the surfaces of platelets or endothelial cells, which have been activated (“stimulated”) during thrombogenic response (e.g. by stimulation of thrombin). The surfaces of these activated cells typically show a significantly higher expression level of these surface molecules than non-activated platelets or endothelial cells. A particular example for such surface molecules is. P-selectin (CD62p). Activated endothelial cells or activated platelets are abundant near the site of vascular damage and thus P-selectin represent “vascular damage related biological structures”.

Other examples for vascular damage related biological structures of this invention are lysosome-associated membrane protein (CD63) (Joern A. Zeller, et al. “Circulating platelets show increased activation in patients with acute cerebral ischemia” Thromb Haemost., Vol. 81, p. 373-377, 1999) or CD40L (Patrick Andre et al. “CD40L stabilizes arterial thrombi by a beta₃ integrin-dependent mechanism” Nature Medicine, Vol. 8, No. 3, p. 247-252, March 2002) or glycoprotein 1b alpha (Janette Burgess et al: “Physical Proximity and Functional Association of Clycoprotein 1b alpha and Protein-disulfide Isomerase on the Platelet Plasma Membrane” The Journal of Biological Chemistry, Vol. 275, No. 13, p. 9758-9766, 2000) or proteindisulfideisomerase (Zaverio Ruggeri “Platelets in atherothrombosis” Nature Medicine, Vol. 8, No. 11, p. 1227-1234, November 2002).

The plasminogen activator can be any type of plasminogen activator or analog, fragment, derivative, or variant thereof. In an embodiment it is preferred to use a plasminogen activator either with an increased fibrin specificity compared to native t-PA or with a modification/deletion of the kringle 2 domain. It is particularly preferred to use the plasminogen activator Desmoteplase (DSPA) originally derived from the vampire bat Desmodus rotundus or parts thereof. Preferably the DSPA comprises the serine protease domain and at least one other domain selected from the group consisting of the finger domain, the EGF domain, and the kringle domain, or analogs, fragments, derivatives, or variants thereof.

The thrombolytic fusion protein of this invention targets and binds to vascular damage related biological structures, e.g. to the surface of activated platelets in acute arterial thrombi, therewith generating high local concentrations of a plasminogen activator at the site of a freshly formed vascular damage (e.g. a platelet-rich thrombus), allowing for a reduction in the systemic dose of the thrombolytic agent, and thereby minimizing the lytic effects on older fibrin-rich clots and achieving a wider therapeutic ratio. The fusion protein is useful in the treatment of arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus and deep vein thrombosis, pulmonary embolism, and acute ischemic stroke.

In another aspect, the invention provides pharmaceutical compositions including the subject thrombolytic fusion proteins.

In another aspect, the invention provides for a method for inducing thrombolysis in a patient, comprising administering a therapeutically effective amount of the thrombolytic fusion protein to said patient.

In another aspect, the invention relates to a method for treating and preventing arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus and deep vein thrombosis, pulmonary embolism, and acute ischemic stroke in a patient, comprising administering a therapeutically effective amount of the thrombolytic fusion protein to said patient.

In another aspect, the invention relates to a method for treating acute ischemic stroke in a patient more than three, preferably more than 6 or even more that 9 hours after stroke onset.

In yet another aspect, the invention relates to a kit, comprising a targeting protein, which binds to the surface of activated platelets, operably linked to a fibrin-selective plasminogen activator. Alternatively, the kit may comprise polynucleotide sequences encoding components of the thrombolytic fusion protein.

Also disclosed are methods of making the thrombolytic fusion proteins of the invention, both recombinant and synthetic.

### BRIEF DESCRIPTION OF THE DRAWINGS

FIG. 1. Schematic drawings of the HuSZ51-DSPAalpha1 fusion protein and expression plasmids. (A) P-Selectin targeted DSPA fusion protein. The HuSZ51-DSPAalpha1 fusion protein consists of two Ig light chains and two Ig heavy chains. The light chain consists of the variable region (V_{L}) of the SZ51 light chain, followed by the constant region of human Igkappa. The heavy chain consists of the variable region (V_{H}) of the SZ51 heavy chain, followed by the constant regions CH1, CH2 and CH3 of human IgG1 and the mature, secreted form of DSPAalpha1. (B) HuSZ51-DSPAalpha1 expression plasmids. The light chain construct pLNOSZ51VK/Hygro-kappa is a CMV promoter-driven expression plasmid, which includes the selection marker hygromycin. The heavy chain construct pLNOSZ51VHDSPA/Neo is a CMV promoter-driven expression plasmid, which includes the selection marker neomycin.

FIG. 2. Amino acid sequences of HuSZ51-DSPAalpha1. (A) HuSZ51-V_{L}Ckappa light chain (SEQ ID NO:1). The light chain of the HuSZ51-DSPAalpha1 fusion protein consists of a signal peptide (amino acids 1 to 19), the variable region of the SZ51 light chain (amino acids 20 to 128), and the constant region of human Igkappa (amino acids 129 to 235). (B) HuSZ51-V_{H}Cγ₁₋₃-DSPAalpha1 heavy chain (SEQ ID NO:2). The heavy chain of the HuSZ51-DSPAalpha1 fusion protein consists of a signal peptide (amino acids 1 to 19), the variable region of the SZ51 heavy chain (amino acids 20 to 137), the constant regions CH1, CH2 and CH3 of the human IgG1 (amino acids 138 to 467), and the mature, secreted form of DSPAalpha1 (amino acids 468 to 908).

FIG. 3. Analysis of HuSZ51-DSPAalpha1 by SDS-PAGE and Western blotting. (A) SDS-PAGE. Purified HuSZ51-DSPAalpha1 fusion protein, recombinant DSPAalpha1 and human IgG1 were separated on a 4 to 10% SDS-PAGE gel under non-reducing and reducing conditions, and then stained with Coomassie blue. (B) Western blotting. After separation by SDS-PAGE, HuSZ51-DSPAalpha1, DSPAalpha1 and IgG1 were transferred onto a nitrocellulose membrane. DSPAalpha1 was detected by staining with biotinylated anti-DSPA monoclonal 9B3 followed by HRP-Avidin, and IgG1 was detected by staining with HRP-conjugated goat anti-human IgG Fc.

FIG. 4. Specific binding of HuSZ51-DSPAalpha1 to P-selectin. (A) Nitrocellulose membrane binding assay. The indicated amounts of recombinant P-selectin were separated by SDS-PAGE and transferred onto a nitrocellulose membrane. HuSZ51-DSPAalpha1 and HuSZ51 binding to the immobilized P-selectin were detected by staining with HRP-conjugated goat anti-human IgG Fc. (B) ELISA. 96-Well plates, coated with recombinant P-selectin, were incubated with the indicated concentrations of HuSZ51-DSPAalpha1, IgG1 or BSA. Antibody binding to P-selectin was detected by staining with HRP-conjugated Protein L, which binds to the Igkappa light chain.

FIG. 5. HuSZ51-DSPAalpha1 competes with SZ51 for binding to P-selectin. An ELISA-based competitive P-selectin binding assay was used to compare the in vitro P-selectin binding affinities of SZ51 and HuSZ51-DSPAalpha1. 96-Well plates, coated with recombinant P-selectin, were incubated with the indicated concentrations of HuSZ51-DSPAalpha1 or human IgG1, plus a fixed amount of SZ51. Competitive binding of SZ51 to immobilized P-selectin was detected by incubation with peroxidase-conjugated anti-mouse IgG, which binds to SZ51 but not HuSZ51-DSPAalpha1, followed by incubation with TMB/E substrate.

FIG. 6. Specific binding of HuSZ51-DSPAalpha1 to thrombin-activated platelets. (A) Human platelets. 96-Well plates, coated with thrombin-activated platelets, were incubated with the indicated concentrations of HuSZ51-DSPAalpha1, SZ51 or human IgG1. Antibody binding to activated platelets was detected by staining with HRP-conjugated Protein L, which binds to the Igκ light chain. (B) Dog platelets. 96-Well plates, coated with thrombin-activated platelets, were incubated with the indicated concentrations of HuSZ51-DSPAalpha1 or human IgG1. Antibody binding to activated platelets was detected by staining with HRP-conjugated Protein L, which binds to the Igkappa light chain.

FIG. 7. Catalytic activity of HuSZ51-DSPAalpha1 on chromogenic substrates. S-2288 (D-Ile-Pro-Arg-p-Nitroaniline dihydrochloride) is a chromogenic substrate for a large range of serine proteases. S-2765 (alpha-Benzyloxycarbonyl-D-Arg-Gly-Arg-p-Nitroaniline dihydrochloride) is a chromogenic substrate for the serine protease factor Xa. Hydrolysis of p-Nitroaniline from these chromogenic substrates by HuSZ51-DSPAalpha1 and DSPA is detected by the increase in absorbance at 405 nm.

FIG. 8. Fibrinolytic activity of HuSZ51-DSPAalpha1 in clot lysis assays. Plasminogen activators such as DSPAalpha1 convert plasminogen to plasmin, which in turn degrades fibrin. The rate of fibrin degradation, monitored by absorbance at 405 nm, was determined for HuSZ51-DSPAalpha1 (12.5, 25 and 50 nM) and for an equimolar amount of DSPAalpha1 (25, 50 and 100 nM). In the fibrin clot lysis assay (top panel), fibrinogen and plasminogen are mixed with HuSZ51-DSPAalpha1 or DSPAalpha1; these mixtures are then added to thrombin. The plasma clot lysis assay (bottom panel) is similar to the fibrin clot assay, except human plasma is used as source of plasminogen and fibrin.

FIG. 9. Thrombolytic activity of HuSZ51-DSPAalpha1 in platelet-poor and platelet-rich plasma clot lysis assays. The fibrinolytic activities of HuSZ51-DSPAalpha1, DSPAalpha1, and uPA were compared in the plasma clot lysis assay, using either platelet-poor (top panel) or platelet-rich (bottom panel) plasma.

### DETAILED DESCRIPTION OF THE INVENTION

The thrombolytic fusion protein of the present invention is comprised of a targeting protein, which binds to vascular damage related biological structure operably linked to a plasminogen activator, or analogs, fragments, derivatives, or variants thereof.

### DEFINITIONS

In describing the present invention, the following terms are defined as indicated below.

“Vascular damage related biological structure” means all biological structures which are either by their quality or quantity indicative for a vascular damage. This damage is preferably rather fresh, i.e. not older than a few hours. “Structure” in this context means any molecule which can form a binding partner for the targeting protein. Thus the vascular damage related biological structure is the “target molecule” for the targeting protein. One example for a vascular damage related biological structure is P-selectin which is translocated on the surfaces of activated endothelial cells or activated platelets during thrombogenic response. Since the activated platelets or activated endothelial cells are abundant in the vicinity of vascular damage, P-selectin is a vascular damage related biological structures.

“Recombinant proteins or polypeptides” refer to proteins or polypeptides produced by recombinant DNA techniques, i.e., produced from cells, microbial or mammalian, transformed by an exogenous recombinant DNA expression construct encoding the desired protein or polypeptide. Proteins or polypeptides expressed in most bacterial cultures will be free of glycan. Proteins or polypeptides expressed in yeast may have a glycosylation pattern different from that expressed in mammalian cells. Depending on the expression system used the glycosylation and/or the N-terminal processing of the recombinant might differ compared to the native protein or polypeptide.

“Native” or “naturally occurring” proteins or polypeptides refer to proteins or polypeptides recovered from a source occurring in nature. The term “native DSPA” would include naturally occurring DSPA and fragments thereof, and would include post-translational modifications of DSPA and fragments thereof, including, but not limited to, acetylation, carboxylation, glycosylation, phosphorylation, lipidation, acylation, and cleavage.

“Fusion protein” is a protein resulting from the expression of at least two operatively linked heterologous coding sequences. The fusion protein of this invention is comprised of a targeting protein, which binds to a vascular damage related biological structure operably linked to a plasminogen activator or analogs, fragments, derivatives, or variants thereof.

“Targeting protein” is a protein or peptide or any analog, fragment, derivative or variant thereof that binds to another protein (polypeptide) or a protein complex or to any other target molecule. The targeting protein of this invention is preferably a protein that binds to P-selectin on the surface of activated platelets. For example, an anti-P-selectin antibody is a targeting protein of this invention.

A DNA or polynucleotide “coding sequence” is a DNA or polynucleotide sequence that is transcribed into mRNA and translated into a polypeptide in a host cell when placed under the control of appropriate regulatory sequences. The boundaries of the coding sequence are determined by a start codon at the 5′ N-terminus and a translation stop codon at the 3′ C-terminus. A coding sequence can include prokaryotic sequences, cDNA from eukaryotic mRNA, genomic DNA sequences from eukaryotic DNA, and synthetic DNA sequences. A transcription termination sequence will usually be located 3′ to the coding sequence.

“DNA or polynucleotide sequence” is a heteropolymer of deoxyribonucleotides (bases adenine, guanine, thymine, cytosine). DNA or polynucleotide sequences encoding the fusion proteins of this invention can be assembled from synthetic cDNA-derived DNA fragments and short oligonucleotide linkers to provide a synthetic gene that is capable of being expressed in a recombinant DNA expression vector. In discussing the structure of particular double-stranded DNA molecules, sequences may be described herein according to the normal convention of giving only the sequence in the 5′ to 3′ direction along the non-transcribed strand of cDNA.

“Recombinant expression vector or plasmid” is a replicable DNA vector or plasmid construct used either to amplify or to express DNA encoding the fusion proteins of the present invention. An expression vector or plasmid contains DNA control sequences and a coding sequence. DNA control sequences include promoter sequences, ribosome binding sites, polyadenylation signals, transcription termination sequences, upstream regulatory domains, and enhancers. Recombinant expression systems as defined herein will express the fusion proteins upon induction of the regulatory elements.

“Transformed host cells” refer to cells that have been transformed and transfected with exogenous DNA. Exogenous DNA may or may not be integrated (i.e., covalently linked) to chromosomal DNA making up the genome of the cell. In prokaryotes and yeast, for example, the exogenous DNA may be maintained on an episomal element, such as a plasmid, or stably integrated into chromosomal DNA. With respect to eukaryotic cells, a stably transformed cell is one which is the exogenous DNA has become integrated into the chromosome replication. This stability is demonstrated by the ability of the eukaryotic cell lines or clones to produce a population of daughter cells containing the exogenous DNA.

“Plasminogen activator” refers to all proteins or polypeptides, or analogs, fragments, derivatives or variants thereof which convert inactive plasminogen into active plasmin. Examples for plasminogen activators are DSPA (Desmoteplase), t-PA or Urokinase (including any parts or modifications thereof). All modified plasminogen activators which are based either on t-PA, DSPA or Urokinase are summarized as either “t-PA derived plasminogen activators”, “DSPA derived plasminogen activators” or “Urokinase derived plasminogen activators”, respectively. These “derived” plasminogen activators correspond essentially to the native forms of t-PA, DSPA and Urokinase, respectively, although they can carry some deletions regarding protein domains or parts thereof as well as amino acid deletions or substitutions.

“DSPA” refers to the plasminogen activators derived from the vampire bat Desmodus rotundus, which can either be isolated (and purified) or recombinantly or synthetically produced. DSPA includes the mature, secreted (processed) forms of DSPAalpha1, DSPAalpha2, DSPAbeta, and DSPAgamma, and analogs, fragments, derivatives, and variants thereof, as well as fragments of the analogs, derivatives, and variants. The genes encoding native DSPAalpha1, DSPAalpha2, DSPAbeta, and DSPAgamma have been isolated and sequenced from the vampire bat Desmodus rotundus (Kratzschmar, J. et al. (1991), supra; and U.S. Pat. Nos. 6,008,091 and 5,830,849, all of which are herein incorporated by reference). All four forms of DSPA contain a 36 amino acid signal sequence that is cleaved off to form the mature, secreted DSPA. Upon recombinant production and depending on the expression systems used, the N terminal sequences of DSPA may differ due to imprecise processing of the leader sequence. However the biological functions remains untouched.

“DSPA domain or domains” refers to discrete amino acid sequences that can be associated with a particular function or characteristic of DSPA, such as a characteristic tertiary structural unit. DSPA domains include: a finger domain, an EGF domain, a kringle domain, and a serine protease domain (Kratzschmar, J. et al. (1991), supra).

Mature DSPAalpha1 is a 441 amino acid polypeptide organized into four distinct domains: a finger domain (amino acids 6 to 43), an EGF domain (amino acids 43 to 92), a kringle domain (amino acids 92 to 174), and a serine protease domain (amino acids 174 to 441) (Kratzschmar, J. et al., (1991), supra).

Mature DSPAalpha2 is a 441 amino acid polypeptide organized into four distinct domains: a finger domain (amino acids 6 to 43), an EGF domain (amino acids 43 to 92), a kringle domain (amino acids 92 to 174), and a serine protease domain (amino acids 174 to 441) (Kratzschmar, J. et al. (1991), supra).

Mature DSPAbeta is a 395 amino acid polypeptide organized into three distinct domains: an EGF domain (amino acids 5 to 46), a kringle domain (amino acids 46 to 127), and a serine protease domain (amino acids 127 to 395) (Kratzschmar, J. et al. (1991), supra).

Mature DSPAgamma is a 358 amino acid polypeptide organized into two distinct domains: a kringle domain (amino acids 9 to 90) and a serine protease domain (amino acids 90 to 358) (Kratzschmar; J. et al. (1991), supra).

The terms “analog”, “fragment,” “derivative”, and “variant”, when referring to the fusion proteins of the invention or to the targeting protein, plasminogen activator or domain, mean analogs, fragments, derivatives, and variants of the fusion protein, targeting protein, plasminogen activator or domain, which retain substantially the same biological function or activity as described further below.

An “analog” includes a pro-polypeptide, which includes within it the amino acid sequence of the fusion protein of this invention. The active fusion protein of this invention can be cleaved from the additional amino acids that complete the profusion protein molecule by natural in vivo processes or by procedures well known in the art, such as by enzymatic or chemical cleavage. For example, native DSPAalpha1 is naturally expressed as a 477 amino acid pro-polypeptide that is then processed in vivo to release the 441 amino acid active mature polypeptide.

A “fragment” is a portion of the fusion protein, targeting protein, or domain(s), which retains substantially similar functional activity as the fusion protein, targeting protein, or domain(s), as shown in the in vitro assays disclosed herein as described further below.

A “derivative” includes all modifications to the fusion protein, which substantially preserve the functions disclosed herein and include additional structure and attendant function, e.g., PEGylated fusion proteins which have a greater half-life, O-glycosylated fusion proteins modified by the addition of chondroitin sulfate, and biotinylated fusion proteins, as described further below.

“Substantially similar functional activity” and “substantially the same biological function or activity” each means that the degree of biological activity that is within about 30% to 100% or more of that biological activity demonstrated by the polypeptide to which it is being compared when the biological activity of each polypeptide is determined by the same procedure or assay. For example, a fusion protein or DSPA domains that has substantially similar functional activity to DSPAalpha1 is one that, when tested in the catalytic or fibrinolytic assays described in Examples 5 and 6, respectively, demonstrates the ability to hydrolyze chromogenic substrates or lyse clots in vitro. A targeting protein that has substantially similar functional activity to the anti-P-selectin antibody SZ51 is one that, when tested in the binding assays described in Examples 2, 3, and 4, demonstrates the ability to bind to P-selectin or to activated platelets or to compete with SZ51 for P-selectin binding in vitro.

“Similarity” between two polypeptides is determined by comparing the amino acid sequence and its conserved amino acid substitutes of one polypeptide to the sequence of a second polypeptide. Such conservative substitutions include those described in Dayhoff, M. O., ed., The Atlas of Protein Sequence and Structure 5, National Biomedical Research Foundation, Washington, D.C. (1978), and in Argos, P., EMBO J. (1989), Vol. 8, pp. 779-785. For example, amino acids belonging to one of the following groups represent conservative changes:
Ala, Pro, Gly, Gln, Asn, Ser, Thr:
Cys, Ser, Tyr, Thr;
Val, Ile, Leu, Met, Ala, Phe;
Lys, Arg, His;
Phe, Tyr, Trp, His; and
Asp, Glu.

“Mammal” includes humans and domesticated animals, such as cats, dogs, swine, cattle, sheep, goats, horses, rabbits, and the like.

“Therapeutic ratio” or “therapeutic index” as used herein means the amount of a fusion protein of the invention required to produce a certain efficacy in treatment of disease in a mammal, including, for example, time to reperfusion, duration of reperfusion, or prevention of thrombosis, divided by the amount of fusion protein required to cause a particular unwanted, adverse effect in the same mammal, including, for example, bleeding time or expression of surrogate markers of disease. The therapeutic ratio or index of a fusion protein of the invention is at least 2, but preferably at least 5, and more preferably between 10 and 20. The therapeutic ratio or index of a fusion protein of the invention may be increased by virtue of its better lytic efficiency, its lower bleeding risk, or both.

“Therapeutically effective amount” refers to that amount of a fusion protein of the invention which, when administered to a human in need thereof, is sufficient to effect treatment, as defined below, for arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus, deep vein thrombosis, pulmonary embolism, and acute ischemic stroke. The amount of a fusion protein of the invention which constitutes a “therapeutically effective amount” will vary depending on the fusion protein, the condition and its severity, and the age of the human to be treated, but can be determined routinely by one of ordinary skill in the art having regard to his own knowledge and to this disclosure.

“Treating” or “treatment” as used herein covers the treatment of disease-state in a mammal, preferably a human, which disease-state is characterized by uncontrolled coagulation and platelet aggregation within a damaged blood vessel, in which excessive fibrin and platelet clots form in the vessel and block blood flow, causing ischemic damage to vital tissues or organs, and includes:
(i) preventing the condition from occurring in a human, in particular, when such human is predisposed to the condition but has not yet been diagnosed as having it;
(ii) inhibiting the condition, i.e., arresting its development; or
(iii) relieving the condition, i.e., causing regression of the condition.

All other technical terms used herein have the same meaning as is commonly used by those skilled in the art to which the present invention belongs.

### Targeting Protein

The targeting protein of this invention is a protein or polypeptide (or a part thereof) that has the ability to specifically bind to a particular target molecule, which is defined as being typical or specific for a rather fresh vascular damage (see above). An example for a targeting protein according to the invention is P-selectin. The targeting protein then serves to direct the fusion protein to the site of the vascular damage, in particular to the target structure, e.g. a cell or tissue bearing the target molecule.

In one embodiment of this invention, the targeting protein is an antibody that can bind to P-selectin. “Antibody” as used herein includes intact immunoglobulin (“Ig”) molecules, as well as fragments thereof, such as Fab, F(ab′)₂, and Fv, which are capable of binding to an epitope of P-selectin. Typically, at least 6, 9, 10, or 12 contiguous amino acids are required to form an epitope. However, epitopes that involve non-contiguous amino acids may require more, e.g., at least 15, 25, or 50 amino acids.

Typically, an antibody that binds specifically to P-selectin provides a detection signal at least 5-, 10-, or 20-fold higher than a detection signal provided with other proteins when used in an immunochemical assay. Preferably, antibodies that bind specifically to P-selectin do not detect other proteins in immunochemical assays, and can immunoprecipitate P-selectin from solution.

P-selectin can be used to immunize a mammal, such as a mouse, rat, rabbit, guinea pig, monkey, or human to produce polyclonal antibodies. If desired, P-selectin can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface-active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Cornybacterium parvum are especially useful.

Monoclonal antibodies that bind specifically to P-selectin can be prepared using any technique which provides for the production of antibody molecules by continuous cell lines in culture. These techniques include, but are not limited to, the hybridoma technique (Kohler, G. and Milstein, C., Nature (1985), Vol. 256, pp. 495-497), the human B-cell hybridoma technique (Kozbor, D. et al., Immunology Today (1983), Vol. 4, pp. 72-79), and the EBV-hybridoma technique (Cole, S. P. C. et al., in Monoclonal Antibodies and Cancer Therapy, pp. 77-96 (eds., Reisfeld, R. A. and Sell, S., Alan R. Liss, Inc., New York, N.Y., 1985).

In addition, techniques developed for the production of “chimeric antibodies,” the splicing of mouse antibody genes to human antibody genes to obtain a molecule with appropriate antigen specificity and biological activity, can be used (see Morrison, S. L. et al., Proc. Natl. Acad. Sci. USA (1984), Vol. 81, pp. 6851-6855; Neuberger, M. S. et al., Nature (1984), Vol. 312, pp. 604-608; and Takeda, S. et al., Nature (1985), Vol. 314, pp. 452-454). Monoclonal and other antibodies also can be “humanized” to prevent a patient from mounting an immune response against the antibody when it is used therapeutically. Such antibodies may be sufficiently similar in sequence to human antibodies to be used directly in the fusion protein or may require alteration of a few key residues. Amino acid sequence differences between rodent and human antibodies can be minimized by replacing the rodent sequences with their human counterparts by site-directed mutagenesis of individual residues, or by grafting of entire complementarity determining regions. Alternatively, humanized antibodies can be produced using recombinant methods, as described in GB2188638B. Anti-bodies that bind specifically to P-selectin can contain antigen-binding sites that are either partially or fully humanized, as disclosed in U.S. Pat. No. 5,565,332. For the purpose of disclosure of the P-selectin specific antibodies this patent is fully incorporated by reference.

Alternatively, techniques described for the production of single chain antibodies can be adapted using methods known in the art to produce single chain anti-bodies that specifically bind to P-selectin. Antibodies with related specificity, but of distinct idiotypic composition, can be generated by chain shuffling from random combinatorial Ig libraries (Kang, A. S. et al., Proc. Natl. Acad. Sci. USA (1991), Vol. 88, pp. 11120-11123).

Single chain antibodies can also be constructed using a DNA amplification method, such as PCR, using hybridoma cDNA as a template (Thirion, S. et al., Eur. J. Cancer Prev. (1996), Vol. 5, pp. 507-511). Single chain antibodies can be mono- or bi-specific, and can be bivalent or tetravalent. Construction of tetravalent, bi-specific single chain antibodies is taught, for example, in Coloma, M. J. and Morrison, S. L., Natl. Biotechnol. (1991), Vol. 15, pp. 159-163. Construction of bivalent, bi-specific single chain antibodies is taught in Mallender, W. D. and Voss, E. W. Jr., J. Biol. Chem. (1994), Vol. 269, pp. 199-206.

A nucleotide sequence encoding a single chain antibody can be constructed using manual or automated nucleotide synthesis, cloned into an expression construct using standard recombinant DNA methods, and introduced into a cell to express the coding sequence. Alternatively, single chain antibodies can be produced directly using, for example, filamentous phage display technology (Verhaar, M. J. et al., Int. J. Cancer (1995), Vol. 61, pp. 497-501; and Nicholls, P. J. et al., J. Immunol. Meth. (1993), Vol. 165, pp. 81-91).

Antibodies that bind specifically to P-selectin can also be produced by inducing in vivo production in the lymphocyte population or by screening Ig libraries or panels of highly specific binding reagents as disclosed in the literature (Orlandi, R. et al., Proc. Natl. Acad. Sci. USA (1989), Vol. 86, pp. 3833-3837; and Winter, G. and Milstein, C., Nature (1991), Vol. 349, pp. 293-299).

Antibody fragments that contain specific binding sites for P-selectin may be generated by recombinant DNA technology, for example, Fab fragments can be generated by reducing the disulfide bridges of the F(ab′)₂ fragments. Alternatively, Fab expression libraries may be constructed to allow for the rapid and easy identification of monoclonal Fab fragments with the desired specificity (Huse, W. D. et al., Science (1989), Vol. 246, pp. 1275-1281).

The targeting protein of this invention (i.e., antibodies) can be expressed and purified by methods well known in the art. For example, the antibodies can be affinity purified by passage over a column to which P-selectin is bound. The bound antibodies can then be eluted from the column using a buffer with a high salt concentration.

In one preferred embodiment of this invention, the targeting protein is a chimeric mouse-human anti-P-selectin monoclonal antibody, HuSZ51, derived from the murine anti-P-selectin monoclonal antibody, SZ51, which was developed at the Jiangsu Institute of Hematology. In platelet binding and Western blots, SZ51 specifically recognizes human P-selectin present on the surface of thrombin-activated, but not resting, platelets. There are ˜11,000 binding sites per thrombin-activated human platelet, and the affinity of the antibody is ˜4 nM (Wu, G. et al., Nouv. Rev. Fr. Hematol. (1990), Vol. 32, pp. 231-235). SZ51 detects arterial and venous thrombi in radiolabeled imaging studies in experimental animal models and human patients, demonstrating its specificity in vivo (Wu, J. et al., Nucl. Med. Commun. (1993), Vol. 14, pp. 1088-1092).

HuSZ51 was generated by constructing two expression plasmids containing the cDNAs encoding the V_{L} and V_{H} regions of SZ51 5′ to the cDNAs encoding the human Igkappa light chain and IgGgamma1 heavy chain constant regions, respectively (Gu, J. et al., Thromb. Haemost. (1997), Vol. 77, pp. 755-759). In platelet-binding and immunoblotting experiments, HuSZ51 specifically recognizes human P-selectin on the surface of thrombin-activated platelets (Gu, J. et al. (1997), supra).

“Anti-P-selectin” refers to a monoclonal antibody that binds to P-selectin. The antibody may or may not interfere with the biological activities of P-selectin, including, but not limited to, the ability to bind to P-selectin-glycoprotein-ligand-1 (PSGL-1), to bind to the glycoprotein Ib-IX-V complex, or to mediate the adherence of leukocytes.

As used herein, the term “specifically binds to” refers to the interaction of an antibody and a target polypeptide, in which the interaction is dependent upon the presence of a particular structure (i.e., the antigenic determinant or epitope) on the polypeptide; in other words, the antibody is recognizing and binding to a specific polypeptide structure rather than to proteins in general.

### Plasminogen Activators

Plasminogen activators convert inactive plasminogen into active plasmin. Although all plasminogen activators of the invention activate plasminogen, examples of plasminogen activators may differ in their structural compositions and specific biological—and thus pharmacological—properties. For all plasminogen activators with essentially the same structural properties (in particular domains/amino acid sequence) as native t-PA the term “t-PA derived” plasminogen activators is used.

In a preferred embodiment of the invention plasminogen activators are used as part of the fusion protein which are characterized by an increased fibrin specificity. Preferably the capacity of these plasminogen activators to activate plasmin is enhanced in the presence of fibrin by more than 650 fold compared to native t-PA. Modifications of the amino acid sequence which render plasminogen activator, in particular t-PA derived plasminogen activators, more fibrin specific are disclosed in EP 1 308 166 A1, the disclosure of which is fully incorporated by reference.

Preferred mutations enhancing the fibrin specificity of t-PA include the following t-PA mutants: t-PA/R275E; t-PA/R275E, F305H; t-PA/R275E, F305H, A292S. Furthermore t-PA variants can be used which carry a point mutation of Asp194 or of an aspartate in a homologue position, leading to a reduced stability of the catalytically active conformation of the plasminogen activating factor in the absence of fibrin. Therefore Asp194 can be substituted by glutamate or asparagine. In another example the plasminogen activator comprises at least one mutation in its autolysis loop, which reduces the functional interactions between plasminogen and plasminogen activating factor in the absence of fibrin. Relevant mutations of the autolysis loop are e.g. in the amino acid positions 420 to 423 of wild type t-PA or homologous positions, which can be substituted as follows: L420A, L420E, S421G, S421E, P422A, P422G, P422E, F423A and F423E.

In yet another embodiment these t-PA variants can be modified in order to prevent cleavage/catalysis by plasmin. These mutations (e.g. a glutamate substitution) can be located at the amino acid position 15 or 275 of the native t-PA or at positions homologous to those.

Furthermore plasminogen activators derived from t-PA can be applied in this invention which are modified in their kringle 2 domain as compared to native t-PA. These modifications either comprise amino acid substitution(s) or even a full deletion of kringle 2 or parts thereof. The lysine binding sites of these modified t-PA variants are preferably modified in order to reduce the lysine binding capacity of the plasminogen activator. Preferred modified t-PA variants are disclosed in WO 2005/026341, which is fully incorporated herewith. A preferred amino acid substitution is D236N.

The plasminogen activators of this invention can have an LHST amino acid sequence at the plasmin activation site. Furthermore the linking sequence between the remaining kringle (in case of full deletion of kringle 2) and the subsequent cysteine bridge is preferably the amino a acid sequence SKAT.

The amino acid sequence of especially preferred plasminogen activators according to the invention are given in FIGS. 10 to 15 (SEQ ID NO. 3 to SEQ ID No 8). Plasminogen activators with an amino acid sequence of least 70%, preferably with an identity of in between 80 to 90%, particularly preferred an identity of 95% can be use in this invention as well.

### DSPA

As outlined above one particularly preferred plasminogen activator of this invention is DSPA. The full length DSPA includes a finger domain, an EGF domain, a kringle domain, and a serine protease domain (Kratzschmar, J. et al. (1991), supra). In preferred embodiments of this invention, the DSPA portion of the fusion protein comprises at least the serine protease domain, preferably in combination with one or more of the other DSPA domains.

The full-length DNA sequences encoding the DSPA proteins from the vampire bat Desmodus rotundus facilitate the preparation of genes and are used as starting point to construct DNA sequences encoding DSPA peptides, fusion proteins containing DSPA, and fragments or peptides of DSPA.

The full-length genes for DSPAalpha1, DSPA alpha2, DSPAbeta, and DSPAgamma have been isolated and sequenced from the vampire bat Desmodus rotundus (Kratzschmar, J. et al. (1991), supra; and U.S. Pat. Nos. 6,008,091 and 5,830,849, all of which are herein incorporated by reference). The full-length DSPAalpha1, DSPA alpha2, DSPAbeta, and DSPAgamma cDNAs can be prepared by several methods. Oligonucleotide probes, specific to these genes, can be synthesized using the published cDNA sequences. For example, messenger RNA prepared from the salivary glands of the vampire bat Desmodus rotundus provides suitable starting material for the preparation of cDNA. Methods for making cDNA libraries and screening cDNA libraries with oligonucleotide probes are well known (see e.g., Sambrook, J. E. et al., Molecular Cloning: A Laboratory Manual, Cold Spring Harbor Laboratory, New York, N.Y., 1989). Alternatively, full-length cDNA for DSPA can be prepared from a cDNA library using specific primers and the polymerase chain reaction (PCR). Methods of isolating cDNA sequences by PCR are well known to those skilled in the art.

### Fusion Protein

The thrombolytic fusion protein of this invention comprises a targeting protein, which preferably binds to the surface of activated platelets, operably linked to a plasminogen activator, or analogs, fragments, derivatives, or variants thereof. In a particular embodiment the plasminogen activator derives from the vampire bat Desmodus rotundus, and comprises the serine protease domain and at least one other domain selected from the group consisting of the finger domain, an EGF domain, and a kringle domain, or analogs, fragments, derivatives, or variants thereof.

In one particularly preferred embodiment, the fusion protein comprises an antibody, or fragment thereof, which binds to P-selectin, operably linked to DSPAalpha1, DSPAalpha2, DSPAbeta, or DSPAgamma, or analogs, fragments, derivatives, or variants thereof.

The fusion protein of the present invention includes, but is not limited to, polypeptides in which the C-terminal portion of a single chain antibody is fused to the N-terminal portion of the plasminogen activator (e.g. DSPA) or an analog, fragment, derivative, or variant thereof, the C-terminal portion of an IgG antibody is fused to the N-terminal portion of the plasminogen activator or an analog, fragment, derivative, or variant thereof, the C-terminal portion of an Fab anti-body is fused to the N-terminal portion of the plasminogen activator or an analog, fragment, derivative, or variant thereof, the N-terminal portion of a single chain antibody is fused to the C-terminal portion of the plasminogen activator or an analog, fragment, derivative, or variant thereof, the N-terminal portion of an IgG antibody is fused to the C-terminal portion of the plasminogen activator or an analog, fragment, derivative, or variant thereof, the N-terminal portion of an Fab antibody is fused to the C-terminal portion of the plasminogen activator or an analog, fragment, derivative, or variant thereof, more than one single chain antibody is fused to both the N-terminal and the C-terminal portions of the plasminogen activator or an analog, fragment, derivative, or variant thereof, more than one IgG antibody is fused to both the N-terminal and the C-terminal portions of the plasminogen activator or an analog, fragment, derivative, or variant thereof, more than one Fab antibody is fused to both the N-terminal and the C-terminal portions of the plasminogen activator or an analog, fragment, derivative, or variant thereof, more than the plasminogen activator or an analog, fragment, derivative, or variant thereof is fused to both the N-terminal and the C-terminal portions a single chain antibody, more than one plasminogen activator or an analog, fragment, derivative, or variant thereof is fused to both the N-terminal and the C-terminal portions an IgG antibody, more than one plasminogen activator or an analog, fragment, derivative, or variant thereof is fused to both the N-terminal and the C-terminal portions an Fab antibody, one or more than one plasminogen activator or an analog, fragment, derivative, or variant thereof is fused to both the N-terminal and the C-terminal portions of a dimeric single chain antibody.

The thrombolytic fusion proteins of the invention include the fusion protein of Example 1 (SEQ ID NOs:1 and 2), as well as those fusion proteins having insubstantial variations in sequence therefrom. An “insubstantial variation” would include any sequence, substitution, or deletion variant that maintains substantially at least one biological function of the polypeptides of this invention, preferably fibrin-selective plasminogen activation activity. These functional equivalents may preferably include fusion proteins that have at least about a 90% identity to the fusion protein of SEQ ID NOs:1 and 2, and more preferably at least a 95% identity to the fusion protein of SEQ ID NOs:1 and 2, and still more preferably at least a 97% identity to the fusion proteins of SEQ ID NOs:1 and 2, and also include portions of such fusion proteins having substantially the same biological activity. However, any fusion protein having insubstantial variation in amino acid sequence from the fusion protein of SEQ ID NOs:1 and 2 that demonstrates functional equivalency as described further herein is included in the description of the present invention.

### Analogs, Fragments, Derivatives, and Variants

An analog, fragment, derivative, or variant of the fusion proteins, as well as of the targeting proteins or the plasminogen activator, of the present invention may be: (i) one in which one or more of the amino acid residues are substituted with a conserved or non-conserved amino acid residue (preferably a conserved amino acid residue) and such substituted amino acid residue may or may not be one encoded by the genetic code; or (ii) one in which one or more of the amino acid residues includes a substituent group, or (iii) one in which the mature fusion protein is fused with another compound, such as a compound to increase the half-life of the fusion protein-(for example, polyethylene glycol), or (iv) one in which additional amino acids are fused to the mature fusion protein, such as a leader or secretory sequence or a sequence which is employed for purification of the mature fusion protein, or (v) one in which the polypeptide sequence is fused with a larger polypeptide, i.e., human albumin, an antibody or Fc, for increased duration of effect. Such analogs, fragments, derivatives, and variants are deemed to be within the scope of those skilled in the art from the teachings herein.

Preferably, the derivatives of the present invention will contain conservative amino acid substitutions (defined further below) made at one or more predicted, preferably nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-conservative substitutions would not be made for conserved amino acid residues or for amino acid residues residing within a conserved protein domain. Fragments or biologically active portions include polypeptide fragments suitable for use as a medicament, as a research reagent, and the like. Fragments; include peptides comprising amino acid sequences sufficiently similar to or derived from the amino acid sequences of a fusion protein of this invention and exhibiting at least one activity of that polypeptide, but which include fewer amino acids than the full-length polypeptides disclosed herein. Typically, biologically active portions comprise a domain or motif with at least one activity of the polypeptide. A biologically active portion of a polypeptide can be a peptide that is, for example, 5 or more amino acids in length. Such biologically active portions can be prepared synthetically or by recombinant techniques and can be evaluated for one or more of the functional activities of a polypeptide of this invention by means disclosed herein and/or well known in the art.

Moreover, preferred derivatives of the present invention include mature fusion proteins that have been fused with another compound, such as a compound to increase the half-life of the polypeptide and/or to reduce potential immunogenicity of the polypeptide (for example, polyethylene glycol, “PEG”). The PEG can be used to impart water solubility, size, slow rate of kidney clearance, and reduced immunogenicity to the fusion protein. See e.g., U.S. Pat. No. 6,214,966. In the case of PEGylations, the fusion of the fusion protein to PEG can be accomplished by any means known to one skilled in the art. For example, PEGylation can be accomplished by first introducing a cysteine mutation into the fusion protein, followed by site-specific derivatization with PEG-maleimide. The cysteine can be added to the C-terminus of the peptides. See, e.g., Tsutsumi, Y. et al., Proc. Natl. Acad. Sci. USA (2000), Vol. 97, pp. 8548-8553. Another modification that can be made to the fusion protein involves biotinylation. In certain instances, it may be useful to have the fusion protein biotinylated so that it can readily react with streptavidin. Methods for biotinylation of proteins are well known in the art. Additionally, chondroitin sulfate can be linked with the fusion protein.

Variants of the fusion proteins, targeting proteins, and plasminogen activators of this invention include polypeptides having an amino acid sequence sufficiently similar to the amino acid sequence of the original fusion proteins, targeting proteins, and plasminogen activators. The term “sufficiently similar’ means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, preferably about 75% through 98%, identical are defined herein as sufficiently similar. Preferably, variants will be sufficiently similar to the amino acid sequence of the preferred fusion proteins of this invention. Variants include variants of fusion proteins encoded by a polynucleotide that hybridizes to a polynucleotide of this invention or a complement thereof under stringent conditions. Such variants generally retain the functional activity of the fusion proteins of this invention. Libraries of fragments of the polynucleotides can be used to generate a variegated population of fragments for screening and subsequent selection. For example, a library of fragments can be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the fusion proteins of this invention.

Variants include fusion proteins, as well as targeting proteins and plasminogen activators, that differ in amino acid sequence due to mutagenesis. Variants that have plasminogen activation activity (which is preferably fibrin-selective) can be identified by screening combinatorial libraries of mutants, for example truncation or point mutants, of the fusion proteins or plasminogen activators of this invention using the catalytic and fibrinolytic assays described in Examples 5 and 6, respectively. Variants that have P-selectin binding activity can be identified by screening combinatorial libraries of mutants, for example truncation or point mutants, of the fusion proteins or targeting proteins of this invention using the P-selectin binding assays described in Examples 2, 3, and 4.

A variegated library of variants can be generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential variant amino acid sequences is expressible as individual polypeptides, or, alternately, as a set of larger fusion proteins (for example, for phage display) containing the set of sequences therein. There are a variety of methods that can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential variant sequences. Methods for synthesizing degenerate oligonucleotides are known in the art (see, e.g., Narang, S. A., Tetrahedron (1983), Vol. 39, p. 3; Itakura, K. et al., Annu. Rev. Biochem. (1984a), Vol. 53, pp. 323-356; Itakura, K. et al., Science (1984b), Vol. 98, pp. 1056-1063; and Ike, Y. et al., Nucleic Acid Res. (1983), Vol. 11, pp. 477-488).

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of fusion proteins, as well as targeting proteins and plasminogen activators, for fibrin-selective plasminogen activation activity or for P-selectin binding activity. The most widely used techniques, which are amenable to high throughput analysis for screening large gene libraries typically include cloning the gene library into replicable expression vectors, transforming appropriate cells with the resulting library of vectors and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify the desired variants.

### Producing Fusion Proteins

The fusion proteins of this invention are produced by fusing the targeting protein to, or otherwise binding it to, the plasminogen activator(s), or analogs, fragments, derivatives or variants thereof, by any method known to those skilled in the art. The two components may be chemically bonded together by any of a variety of well-known chemical procedures. For example, the linkage may be by way of heterobifunctional cross-linkers, e.g., SPDP, carbodiimide, glutaraldehyde, or the like.

In a more preferred embodiment, the targeting protein of this invention can be fused to the DSPA domain(s) by recombinant means such as through the use of recombinant DNA techniques to produce nucleic acids which encode both the targeting protein and the polypeptide encoding the plasminogen activator(s), and expressing the DNA sequences in a host cell such as E. coli or a mammalian cell. The DNAs encoding the fusion protein may be cloned in cDNA form by any cloning procedure known to those skilled in the art. See, for example, Sambrook, J. E. et al. (1989) supra.

In the case where the targeting protein is a single chain antibody, once a DNA sequence has been identified that encodes a Fv region which when expressed shows specific binding activity, fusion proteins comprising that Fv region may be prepared by methods known to one of skill in the art. Thus, for example, Chaudhary, V. K. et al., Nature (1989), Vol. 339, pp. 394-397; Batra, J. K. et al., J. Biol. Chem. (1990), Vol. 265, pp. 15198-15202; Batra, J. K. et al., Proc. Natl. Acad. Sci. USA (1989), Vol. 86, pp. 8545-8549; and Chaudhary, V. K. et al., Proc. Natl. Acad. Sci. USA (1990), Vol. 87, pp. 1066-1070, all incorporated by reference, describe the preparation of various single chain antibody fusion proteins. The Fv region may be fused directly to the plasminogen activator (s) or may be joined via a linker sequence. The linker sequence may be present simply to provide space between the targeting moiety and the plasminogen activator(s) or to facilitate mobility between these regions to enable them to each attain their optimum conformation. The DNA sequence comprising the connector may also provide sequences (such as primer or restriction sites) to facilitate cloning or may preserve the reading frame between the sequence encoding the targeting moiety and the sequence encoding the plasminogen activator(s). The design of such connector peptides will be well known to those of skill in the art.

In the present invention, linker sequences can be used for linking the anti-P-selectin antibody, or analog, fragment, derivative, or variant thereof, to the plasminogen activator(s) or analog, fragment, derivative, or variant thereof. Linker sequences can also be used for linking the heavy and light chain domains of the single chain antibody. It will be apparent that linker sequences from 0 to 15 amino acids may be used. It will be apparent to those having skill in the art that many different linker sequences may be used and still result in a fusion protein that retains fibrinolytic activity, fibrin selectivity, P-selectin binding activity, and activated platelet binding activity. Modifications of the linker sequences may be aimed at maximizing the enhancement of fibrinolytic activity, fibrin selectivity, P-selectin binding activity, and/or activated platelet binding activity.

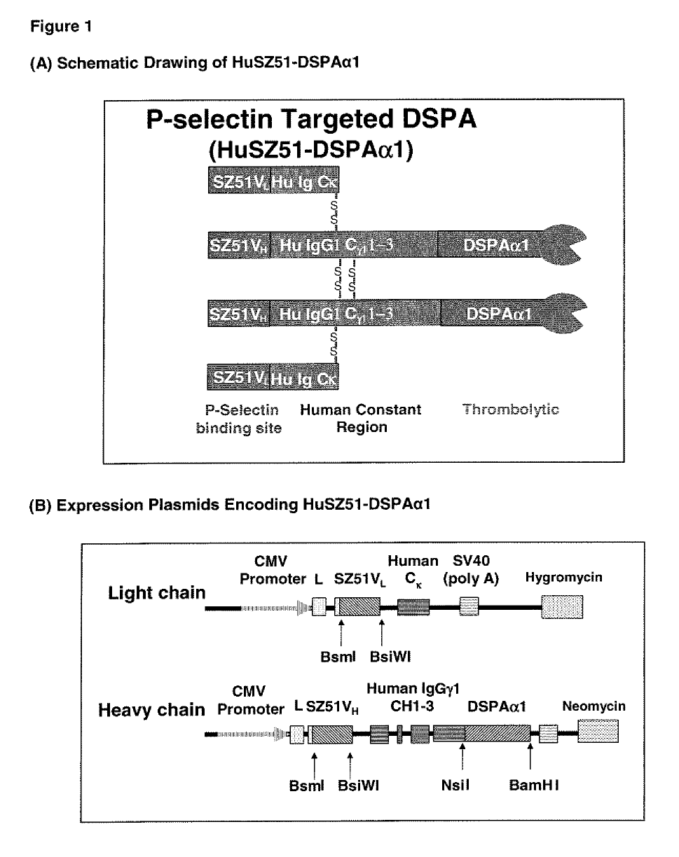

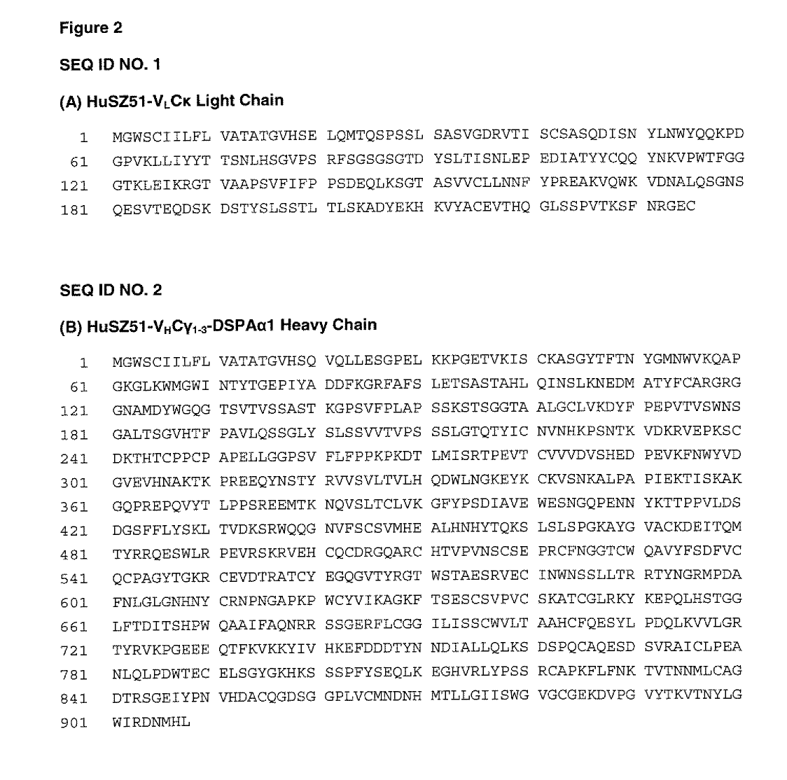

In a preferred embodiment of the present invention, an anti-P-selectin-DSPAalpha1 fusion protein was generated by constructing an expression vector in which the cDNA encoding the mature DSPAalpha1 was inserted 3′ to the SZ51-V_{H}-human IgG1 CH3 region (Gu, J. et al., (1997), supra). The resulting expression plasmid was co-transfected with the expression plasmid encoding the SZ51-V_{L}-human Igκ chimeric protein to generate HuSZ51-DSPAalpha1. Schematic drawings of the resultant fusion protein and the light chain and heavy chain expression plasmids are depicted in FIG. 1. The HuSZ51-DSPAalpha1 molecule contains two Ig light and two Ig heavy chains. The light chain consists of the V_{L} region of SZ51 and the constant region of human Igκ. The heavy chain consists of the V_{H} region of SZ51, constant regions 1 through 3 of human IgG1, and the full-length DSPAalpha1. The amino acid sequences of the two polypeptide chains of HuSZ51-DSPAalpha1 are depicted in FIG. 2 (SEQ ID NOs:1 and 2).

A similar construct was made using the full-length human uPA cDNA, to generate the fusion protein, HuSZ51-uPA (Wan, H. et al., Thromb. Res. (2000), Vol. 97, pp. 133-141). In this case, affinity purified HuSZ51-uPA inhibits binding of the parental murine monoclonal antibody SZ51 to thrombin-activated human platelets, demonstrating that an anti-P-selectin-plasminogen activator fusion protein can retain platelet-binding activity in vitro (Wan, H. et al. (2000), supra).

HuSZ51-DSPAalpha1 was generated by first inserting the cDNAs encoding the V_{L} and V_{H} regions of SZ51 into expression vectors containing the human Igκ light and IgG1 heavy chain constant regions, respectively. The light chain construct, pLNOSZ51VK/Hygro, is a viral CMV promoter-driven expression plasmid, which includes the selection marker hygromycin. To construct this plasmid, a DNA fragment encoding the V_{L} region of SZ51, flanked by BsmI and BsiWI restriction enzyme recognition sites, was generated by standard PCR technology, digested with Bsm I and BsiWI, and inserted between the BsmI and BsiWI sites of the plasmid pLNO/Kappa/Hygromycin (Norderhaug, L. et al., J. Immunol. Meth. (1997), Vol. 204, pp. 77-87). The resultant light chain construct, consisting of the SZ51-V_{L} region and human Igκ constant region, is depicted in FIG. 1B. The heavy chain construct, pLNOSZ51VH/Neo, is a viral CMV promoter-driven expression plasmid, which includes the selection marker neomycin. To construct this plasmid, a DNA fragment containing the V_{H} region of SZ51, flanked by BsmI and a BsiWI restriction enzyme recognition sites, was generated by standard PCR technology, digested with BsmI and BsiWI, and inserted between the BsmI and BsiWI sites of the plasmid pLNOH/IgG1/Neo (Norderhaug, L. et al. (1997), supra). Next, a DNA fragment was generated by overlapping PCR containing the CH3 domain of IgG1 (spanning a NsiI restriction enzyme site) fused directly to the mature, secreted form of DSPAalpha1 (flanked by a BamHI restriction enzyme site). The resultant PCR fragment was digested with NsiI and BamHI, and then inserted between the NsiI and BamHI sites of the plasmid pLNOSZ51VH/Neo. The resultant chimeric heavy chain-DSPA fusion construct, pLNOSZ51VHDSPA/Neo, consisting of the SZ51-V_{H} region, human IgG1 constant region, and mature, secreted form of DSPAalpha1, is depicted in FIG. 1B.

Using these methods, other anti-P-selectin-DSPAalpha1 fusion protein expression plasmids were generated, including HuSZ51 Fab′-DSPAalpha1, which lacks the CH2 and CH3 domains in HuSZ51-DSPAalpha1; and scFvSZ51-DSPAalpha1, which is a single chain antibody containing (from N- to C-terminus) the SZ51-V_{L} region followed by the SZ51-V_{H} region and the mature, secreted form of DSPAalpha1. Similar methods are used to generate other fusion protein expression plasmids of the invention.

### Expression and Purification of Fusion Proteins

There are several ways to express the recombinant fusion proteins in vitro, including E. coli, baculovirus, yeast mammalian cells, or other expression systems. Methods for the expression of cloned genes in bacteria are well known. To obtain high level expression of a cloned gene in a prokaryotic system, it is essential to construct expression vectors that contain, at the minimum, a strong promoter to direct mRNA transcription termination. Examples of regulatory regions suitable for this purpose are the promoter and operator region of the E. coli beta-glucosidase gene, the E. coli tryptophan biosynthetic pathway, or the leftward promoter from phage lambda. The inclusion of selection markers in DNA plasmids transformed in E. coli is useful. Examples of such markers include the genes specifying resistance to ampicillin, tetracycline, or chloramphenicol.

Of the higher eukaryotic cell systems useful for expression of the fusion proteins of the invention there are numerous cell systems to select from. Illustrative examples of mammalian cell lines include, but are not limited to, RPMI 7932, VERO, and HeLa cells, Chinese hamster ovary (“CHO”) cell lines, W138, BHK, COS-7, C127, or MDCK cell lines. Cells suitable for use in this invention are commercially available from the ATCC. Illustrative non-mammalian eukaryotic cell lines include but are not limited to Spodoptera frugiperda and Bombyx mori.

Post-translational modifications, such as glycosylation, do not occur in the prokaryotic cell expression system E. coli. In addition, proteins with complex disulfide patterns are often misfolded when expressed in E. coli. With the prokaryotic system, the expressed protein is either present in the cell cytoplasm in an insoluble form so-called inclusion bodies, found in the soluble fraction after the cell has lysed, or is directed into the periplasm by the addition of appropriate secretion signal sequences. If the expressed protein is in insoluble inclusion bodies, solubilization and subsequent refolding of the inclusion bodies are usually required.

Many prokaryotic expression vectors are known to those of skill in the art and are commercially available, such as pKK223-3 (Pharmacia Fine Chemicals, Uppsala, Sweden), pKK233-2 (Clontech, Palo Alto, Calif., USA), and pGEM1 (Promega Biotech, Madison, Wis., USA).

Promoters commonly used in recombinant microbial expression systems include the beta-lactamase (penicillinase) and lactose promoter system (Chang, A. C. et al., Nature (1978), Vol. 275, pp. 617-624; and Goeddel, D. V. et al., Nature (1979), Vol. 281, pp. 544-548), the tryptophan (trp) promoter system (Goeddel, D. V. et al., Nucl. Acids Res. (1980), Vol. 8, pp. 4057-4074) and tac promoter (Sambrook, J. E. et al., (1989), supra). Another useful bacterial expression system employs the lambda phage pL promoter and cits857 thermoinducible repressor (Bernard, H. U. et al., Gene (1979), Vol. 5, pp. 59-76; and Love, C. A. et al., Gene (1996), Vol. 176, pp. 49-53). Recombinant fusion proteins may also be expressed in yeast hosts such as Saccharomyces cerevisiae. It usually provides the ability to do various post-translational modifications. The expressed fusion protein can be secreted into the culture supernatant where not many other proteins reside, facilitating purification. Yeast vectors for expression of the fusion proteins in this invention contain certain requisite features. The elements of the vector are generally derived from yeast and bacteria to permit propagation of the plasmid in both. The bacterial elements include an origin of replication and a selectable marker. The yeast elements include an origin of replication sequence, a selectable marker, a promoter, and a transcriptional terminator.

Suitable promoters in yeast vectors for expression include the promoters of the TRP1 gene, the ADH1 or ADHII gene, the acid phosphatase (PH03 or PH05) gene, the isocytochrome gene, or promoters-involved with the glycolytic pathway, such as the enolase, pyruvate kinase, hexokinase, glyceraldehyde-3-phosphate dehydrogenase (GADPH), 3-phosphoglycerate kinase (PGK), triosephosphate isomerase, and phosphoglucose isomerase promoters (Hitzeman, R. A. et al., J. Biol. Chem. (1980), Vol. 255, pp. 12073-12080; Hess, B. et al., J. Adv. Enzyme Reg. (1968), Vol. 7, pp. 149-167; and Holland, M. J. and Holland, J. P., Biochemistry (1978), Vol. 17, pp. 4900-4907).

Commercially available yeast vectors include pYES2, pPIC9 (Invitrogen, San Diego, Calif.), Yepc-pADH2a, pYcDE-1 (Washington Research, Seattle, Wash.), pBC102-K22 (ATCC # 67255), and YpGX265GAL4 (ATCC # 67233). Mammalian cell lines including, but not limited to, COS-7, L cells, C127, 3T3, CHO, HeLa, BHK, CHL-1, NSO, and HEK293 can be employed to express the recombinant fusion proteins in this invention. Recombinant proteins produced in mammalian cells are normally soluble and glycosylated, and have authentic N-termini. The mammalian expression vectors may contain non-transcribed elements, such as an origin of replication, promoter, and enhancer, and 5′ or 3′ nontranslated sequences such as ribosome binding sites, a polyadenylation site, acceptor site and splice donor, and transcriptional termination sequences. Promoters for use in mammalian expression vectors usually are, for example, viral promoters such as polyoma, adenovirus, HTLV, simian virus 40 (“SV 40”), and human cytomegalovirus (“CMV”).

Depending on the expression system and host selected, a homogeneous recombinant fusion protein can be obtained by using various combinations of conventional chromatography used for protein purification. These include: immunoaffinity chromatography, reverse phase chromatography, cation exchange chromatography, anion exchange chromatography, hydrophobic interaction chromatography, gel filtration chromatography, and HPLC. If the expression system secretes the fusion protein into the growth media, the protein can be purified directly from the media. If the fusion protein is not secreted, it is isolated from cell lysates. Cell disruption can be done by any conventional method, including freeze-thaw cycling, sonication, mechanical disruption, or use of cell lysing agents.

An anti-P-selectin-DSPA fusion protein of the invention was expressed and purified using standard mammalian gene expression and purification technology. In a preferred embodiment of this invention, the mammalian expression constructs were transfected into CHO cells. When CHO cells are used, neomycin or hygromycin may be included as the eukaryotic selection marker.

The chimeric light chain construct, pLNOSZ51VK/Hygro, and the chimeric heavy chain-DSPA fusion construct, pLNOSZ51VHDSPA/Neo, were co-transfected into CHO cells using a liposome-mediated (Lipofectin 2000) method. The transfected CHO cells were selected using 500 μg/ml neomycin and 600 μg/ml hygromycin in HAMS/F-12 medium. One hundred and twelve antibiotic-resistant CHO clones were picked up and screened for the expression of the HuSZ51-DSPAalpha1 fusion protein using an ELISA. Six out of 112 clones expressed the protein at levels ranging from 3 mg/L to 6 mg/L, and the expression levels were maintained for more than 10 cell passages. Three clones, #62, #70 and #87, were used for scale-up cell culture (cell factory) to produce conditioned medium for protein purification.

The conditioned media from CHO clones secreting HuSZ51-DSPAalpha1 was first filtered through a 0.22 μm filter, and then concentrated 10- to 20-fold using a 10 kDa molecular weight cutoff membrane (2 Millipore Pellicon Membranes on the Ultrafiltration Prep System). The concentrated material was then loaded on a Protein A column equilibrated with 50 mM sodium citrate pH 6.5, 300 mM NaCl. When fully loaded, the column was washed with 10 column volumes of the same buffer, and protein was eluted with 50 mM sodium citrate pH 2.7, 300 mM NaCl. The eluate was neutralized with 1 M Tris-HCl, pH 8.0, and the pH of the eluate was maintained between pH 5.0 and 7.0. To eliminate the small amount of contaminating bovine IgG, the Protein A column eluate was loaded onto a cation exchange SP-Fractogel column equilibrated with 20 mM sodium acetate pH 5.0, 100 mM in NaCl, and then eluted with a linear gradient to 0.9 M NaCl at the flow rate was 3 mL/min over 20 min. The second elution peak, which contained the HuSZ51-DSPAalpha1 fusion protein, was collected. The SP-Fractogel column eluate was subjected to further protein purification through a Sephacryl-300 HR gel filtration column to eliminate any protein aggregates.

Using these methods, other anti-P-selectin-DSPAalpha1 fusion proteins were expressed and purified, including HuSZ51Fab′-DSPAalpha1, which lacks the CH2 and CH3 domains in HuSZ51-DSPAalpha1; and scFvSZ51-DSPAalpha1, which is a single chain antibody containing (from N- to C-terminus) the SZ51-V_{L} region followed by the SZ-51-V_{H} region and the mature, secreted form of DSPAalpha1. Similar methods are used to express and purify other fusion proteins of the invention.

### Pharmaceutical Compositions

The invention also provides pharmaceutical compositions that can be administered to a patient to achieve a therapeutic effect. Pharmaceutical compositions of this invention can be prepared for administration by combining a fusion protein, having the desired degree of purity and the pharmaceutically effective amount, with physiologically acceptable carriers.

The fusion proteins of the present invention can be used in pharmaceutical compositions, for intravenous, subcutaneous, intramuscular, or intrathecal administration. Thus, the above described polypeptides preferably will be combined with an acceptable sterile pharmaceutical carrier, such as five percent dextrose, lactated Ringer's solution, normal saline, sterile water, or any other commercially prepared physiological buffer solution designed for intravenous infusion. It will be understood that the selection of the carrier solution, and the dosage and administration of the composition, will vary with the subject and the particular clinical setting, and will be governed by standard medical procedures.

In accordance with the methods of the present invention, these pharmaceutical compositions may be administered in amounts effective to inhibit the pathological consequences associated with excessive fibrin and platelet clots in blood vessels of the subject.

Administration of the fusion protein may be by bolus intravenous injection, by constant intravenous infusion, or by a combination of both routes. Alternatively, or in addition, the fusion protein mixed with appropriate excipients may be taken into the circulation via an intramuscular site.

The recombinant fusion proteins and pharmaceutical compositions of this invention are useful for parenteral, topical, intravenous, oral, or local administration. The pharmaceutical compositions can be administered in a variety of unit dosage forms depending upon the method of administration. For example, unit dosage forms can be administered in the form including, but not limited to, tablets, capsules, powder, solutions, and emulsions.

The recombinant fusion proteins and pharmaceutical compositions of this invention are particularly useful for intravenous administration. The compositions for administration will commonly comprise a solution of the fusion protein dissolved in a pharmaceutically acceptable carrier, preferably in an aqueous carrier. A variety of aqueous carriers can be used, e.g., buffered saline and the like. These solutions are sterile and generally free of undesirable matter. The compositions may be sterilized by conventional, well known sterilization techniques.

A typical pharmaceutical composition for intravenous administration can be readily determined by one of ordinary skill in the art. The amounts administered are clearly protein specific and depend on its potency and pharmacokinetic profile. Actual methods for preparing parenterally administrable compositions will be known or apparent to those skilled in the art and are described in more detail in such publications as Remington's Pharmaceutical Science; 18^{th} ed., Mack Publishing Company, Easton, Pa., 1990).

The compositions containing the present fusion proteins or a cocktail thereof (i.e., with other proteins) can be administered therapeutic treatments. In therapeutic applications, compositions are administered to a patient suffering from arterial thrombosis in an amount sufficient to cure or at least partially arrest the disorder. An amount adequate to accomplish this is defined as a “therapeutically effective dose”. Amounts effective for this use will depend upon the severity of the disease and the general state of the patient's health.

Single or multiple administration(s) of the compositions may be administered depending on the dosage and frequency as required and tolerated by the patient. In any event, the composition should provide a sufficient quantity of the proteins of this invention to effectively treat the patient. Generally, depending on the intended mode of administration, the pharmaceutically acceptable compositions will contain about 1% to about 99% by weight of a fusion protein of the invention, and 99% to 1% by weight of a suitable pharmaceutical excipient or carrier. Preferably, the composition will be about 5% to 75% by weight of a fusion protein(s) of the invention, with the rest being suitable pharmaceutical excipients or carriers.

The fusion proteins of the invention, or their pharmaceutically acceptable compositions, are administered in a therapeutically effective amount, which will vary depending upon a variety of factors including the activity of the specific fusion protein employed; the metabolic stability and length of action of the fusion protein; the age, body weight, general health, sex, and diet of the patient; the mode and time of administration; the rate of excretion; the drug combination; the severity of the particular disease-states; and the host undergoing therapy. Generally, a therapeutically effective daily dose is from about 0.14 mg to about 14.3 mg/kg of body weight per day of a fusion protein of the invention, preferably, from about 0.7 mg to about 10 mg/kg of body weight per day; and most preferably, from about 1.4 mg to about 7.2 mg/kg of body weight per day. For example, for administration to a 70 kg person, the dosage range would be from about 10 mg to about 1.0 gram per day of a fusion protein of the invention, preferably from about 50 mg to about 700 mg per day, and most preferably from about 100 mg to about 500 mg per day.

### Cell and Gene Therapy

A fusion protein of the invention may be employed in accordance with the present invention by expression of such fusion protein in vivo by a method referred to as “cell therapy”. Thus, for example, cells may be engineered with a polynucleotide(s) (DNA or RNA) encoding the fusion protein ex vivo, and the engineered cells are then provided to a patient to be treated with the fusion protein. Such methods are well known in the art. For example, cells may be engineered by procedures known in the art by use of a retroviral particle containing RNA encoding the fusion protein of the present invention.

A fusion protein of the invention may also be employed in accordance with the present invention by expression of such fusion protein in vivo by a method referred to as “gene therapy”. Thus, for example, a virus may be engineered with a polynucleotide(s) (DNA or RNA) encoding the fusion protein, and the engineered virus is then provided to a patient to be treated with the fusion protein. Such methods are well known in the art. For example, recombinant adenoviruses may be engineered by procedures known in the art containing DNA encoding the fusion protein of the present invention.

Local delivery of the fusion proteins of the present invention using cell or gene therapy may provide the therapeutic agent to the target area, the endothelial cells lining blood vessels.

Both in vitro and in vivo cell and gene therapy methodologies are contemplated. Several methods for transferring potentially therapeutic genes to defined cell populations are known. See, e.g., Mulligan, R. C., Science (1993), Vol. 260, pp. 926-932. These methods include: direct gene transfer (see, e.g., Wolff, J. A. et al., Science (1990), Vol. 247, pp. 1465-1468); liposome-mediated DNA transfer (see, e.g., Caplen, N. J. et al., Nature Med. (1995), Vol. 3, pp. 39-46; Crystal, R. G., Nature Med. (1995), Vol. 1, pp. 15-17; Gao, X. and Huang, L., Biochem. Biophys. Res. Comm. (1991), Vol. 79, pp. 280-285); retrovirus-mediated DNA transfer (see, e.g., Kay, M. A. et al., Science (1993), Vol. 262, pp. 117-119; Anderson, W. F., Science (1992), Vol. 256, pp. 808-813); and DNA virus-mediated DNA transfer. Such DNA viruses include adenoviruses (preferably Ad2 or Ad5 based vectors), herpes viruses (preferably herpes simplex virus based vectors), and parvoviruses (preferably “defective” or non-autonomous parvovirus based vectors, more preferably adeno-associated virus based vectors, most preferably MV-2 based vectors). See, e.g., Ali, M. et al., Gene Therapy (1994), Vol. 1, pp. 367-384; U.S. Pat. No. 4,797,368, incorporated herein by reference, and U.S. Pat. No. 5,139,941, incorporated herein by reference.

The choice of a particular vector system for transferring the gene of interest will depend on a variety of factors. One important factor is, the nature of the target cell population. Although retroviral vectors have been extensively studied and used in a number of gene therapy applications, these vectors are generally unsuited for infecting non-dividing cells. In addition, retroviruses have the potential for oncogenicity. However, recent developments in the field of lentiviral vectors may circumvent some of these limitations. See Naldini, L. et al., Science (1996), Vol. 272, pp. 263-267.

Retroviruses from which the retroviral plasmid vectors hereinabove mentioned may be derived include, but are not limited to, Moloney murine leukemia virus, spleen necrosis virus, retroviruses such as Rous sarcoma virus, Harvey sarcoma virus, avian leukosis virus, gibbon ape leukemia virus, human immunodeficiency virus, adenovirus, myeloproliferative sarcoma virus, and mammary tumor virus.

Adenoviruses have the advantage that they have a broad host-range, can infect quiescent or terminally differentiated cells, such as neurons or hepatocytes, and appear essentially non-oncogenic. See, e.g., Ali, M. et al. (1994), supra. Adenoviruses do not appear to integrate into the host genome. Because they exist extrachromosomally, the risk of insertional mutagenesis is greatly reduced. Ali, M. et al. (1994), supra.

Adeno-associated viruses exhibit similar advantages as adenoviral-based vectors. However, AAVs exhibit site-specific integration on human chromosome 19 (Ali, M. et al. (1994), supra).

In a preferred embodiment, the DNA encoding the fusion proteins of this invention is used in cell or gene therapy for cardiovascular diseases including, but not limited to, arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus, deep vein thrombosis, pulmonary embolism, and acute ischemic stroke.

According to this embodiment, cell or gene therapy with DNA encoding fusion proteins of this invention is provided to a patient in need thereof, concurrent with, or immediately after diagnosis.

The skilled artisan will appreciate that any suitable gene therapy vector containing DNA encoding the fusion protein of the invention or DNA encoding analogs, fragments, derivatives, or variants of the fusion proteins of the invention may be used in accordance with this embodiment. The techniques for constructing such a vector are known. See, e.g., Anderson, W. F., Nature (1998), Vol. 392, pp. 25-30; and Verma, I. M. and Somia, N., Nature (1998), Vol. 389, pp. 239-242. Introduction of the fusion protein DNA-containing vector(s) to the target site may be accomplished using known techniques.

The cell or gene therapy vector includes one or more promoters. Suitable promoters which may be employed include, but are not limited to, the retroviral LTR; the SV40 promoter; and the human CMV promoter described in Miller, A. D and Rosman, G. J., Biotechniques (1989), Vol. 7, pp. 980-990, or any other promoter (e.g., cellular promoters such as eukaryotic cellular promoters including, but not limited to, the histone, pol III, and beta-actin promoters). Other viral promoters which may be employed include, but are not limited to, adenovirus promoters, thymidine kinase (“TK”) promoters, and B19 parvovirus promoters. The selection of a suitable promoter will be apparent to those skilled in the art from the teachings contained herein.

The nucleic acid sequence encoding the fusion protein of the present invention is under the control of a suitable promoter. Suitable promoters which may be employed include, but are not limited to, adenoviral promoters, such as the adenoviral major late promoter; or heterologous promoters, such as the CMV promoter; the respiratory syncytial virus promoter; inducible promoters, such as the MMT promoter, the metallothionein promoter; heat shock promoters; the albumin promoter; the ApoAI promoter; human globin promoters; viral thymidine kinase promoters, such as the herpes simplex virus TK promoter; retroviral LTRs (including the modified retroviral LTRs hereinabove described); the beta-actin promoter; and human growth hormone promoter.

The retroviral plasmid vector is employed to transduce packaging cell lines to form producer cell lines. Examples of packaging cells which maybe transfected include, but are not limited to, the PE501, PA317, psi-2, psi-AM, PA12, T19-14X; VT-19-17-H2, psiCRE, psiCRIP, GP+#-86, GP+envAm12, and DAN cell lines as described in Miller, A. D., Hum. Gene Ther. (1990), Vol. 1, pp. 5-14, which is incorporated herein by reference in its entirety. The vector may trans-duce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host. The producer cell line generates infectious retroviral vector particles, which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

A different approach to gene therapy is “transkaryotic therapy” wherein the patient's cells are treated ex vivo to induce the dormant chromosomal genes to produce the protein of interest after reintroduction to the patient. Transkaryotic therapy assumes the individual has a normal complement of genes necessary for activation. Transkaryotic therapy involves introducing a promoter or other exogenous regulatory sequence capable of activating the nascent genes, into the chromosomal DNA of the patient's cells ex vivo, culturing and selecting for active protein-producing cells, and then reintroducing the activated cells into the patient with the intent that they then become fully established. The “gene activated” cells then manufacture the protein of interest for some significant amount of time, perhaps for as long as the life of the patient. U.S. Pat. Nos. 5,641,670 and 5,733,761 disclose in detail this concept, and are hereby incorporated by reference in their entirety.

### Kits

This invention further relates to kits for research or diagnostic purposes. Kits typically include one or more containers containing the fusion proteins of the present invention. In a preferred embodiment, the kits comprise containers containing fusion proteins in a form suitable for derivatizing with a second molecule. In a more preferred embodiment, the kits comprise containers containing the fusion protein of SEQ ID NOs. 1 and 2. Further provided are kits comprising the compositions of the invention, in free form or in pharmaceutically acceptable form. The kit can comprise instructions for its administration. The kits of the invention can be used in any method of the present invention.

In another embodiment, the kits may contain DNA sequences encoding the fusion proteins of the invention. Preferably the DNA sequences encoding these fusion proteins are provided in a plasmid(s) suitable for transfection into and expression by a host cell. The plasmid(s) may contain a promoter (often an inducible promoter) to regulate expression of the DNA in the host cell. The plasmid(s) may also contain appropriate restriction sites to facilitate the insertion of other DNA sequences into the plasmid to produce various fusion proteins. The plasmid(s) may also contain numerous other elements to facilitate cloning and expression of the encoded proteins. Such elements are well known to those of skill in the art and include, for example, selectable markers, initiation codons, termination codons, and the like.

### Therapeutic Indications

Diseases, disorders, or conditions in which thrombus formation plays a significant etiological role include arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus, deep vein thrombosis, pulmonary embolism, and acute ischemic stroke. The fusion proteins of this invention are useful in all of these diseases, disorders, or conditions in which thrombus formation is pathological. By useful it is meant that the fusion proteins are useful for treatment, either to prevent disease or to prevent its progression to a more severe state.

### FURTHER PREFERRED EMBODIMENTS

Of the fusion proteins of the invention as set forth above in the Summary of the Invention, several groups of fusion proteins are particularly preferred.

In one preferred embodiment, the invention relates to a fusion protein comprising an antibody that binds to P-selectin, or analog, fragment, derivative, or variant thereof, operably linked to DSPAalpha1, or analog, fragment, derivative, or variant thereof.

In another preferred embodiment, the invention relates to a fusion protein comprising an antibody that binds to P-selectin, or analog, fragment, derivative, or variant thereof, operably linked to the DSPAalpha1 finger, EGF, and serine protease domains, in any combination thereof, or analog, fragment, derivative, or variant thereof.

In another preferred embodiment, the invention relates to a fusion protein comprising an antibody that binds to P-selectin, or analog, fragment, derivative, or variant thereof, operably linked to the DSPAalpha1 finger, kringle, and serine protease domains, in any combination thereof, or analog, fragment, derivative, or variant thereof.

In another preferred embodiment, the invention relates to a fusion protein comprising an antibody that binds to P-selectin, or analog, fragment, derivative, or variant thereof, operably linked to the DSPAalpha1 finger and serine protease domains, in any combination thereof, or analog, fragment, derivative, or variant thereof.

In another preferred embodiment, the invention relates to a fusion protein comprising a monoclonal antibody that binds to P-selectin, or analog, fragment, derivative, or variant thereof, operably linked to DSPAalpha1, or analog, fragment, derivative, or variant thereof.

In a more preferred embodiment, the invention relates to a fusion protein comprising a chimeric monoclonal antibody that binds to P-selectin, or analog, fragment, derivative, or variant thereof, operably linked to DSPAalpha1, or analog, fragment, derivative, or variant thereof.

In another preferred embodiment, the invention relates to a fusion protein comprising a Fab dimer antibody that binds to P-selectin, or analog, fragment, derivative, or variant thereof, operably linked to DSPAalpha1, or analog, fragment, derivative, or variant thereof.

In another preferred embodiment, the invention relates to a fusion protein comprising a single chain antibody that binds to P-selectin, or analog, fragment, derivative, or variant thereof, operably linked to DSPAalpha1, or analog, fragment, derivative, or variant thereof.

In a more preferred embodiment, the invention relates to a fusion protein comprising HuSZ51, which binds to P-selectin, or analog, fragment, derivative, or variant thereof, operably linked to DSPAalpha1, or analog, fragment, derivative, or variant thereof.

In another more preferred embodiment, the invention relates to a fusion protein comprising HuSZ51, which binds to P-selectin, or analog, fragment, derivative, or variant thereof, operably linked via the heavy chain to DSPAalpha1, or analog, fragment, derivative, or variant thereof.

In another preferred embodiment, the invention relates to a method of treating arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus and deep vein thrombosis, pulmonary embolism, and acute ischemic stroke, comprising administering to a human in need thereof a therapeutically effective amount of a fusion protein comprising HuSZ51, which binds to P-selectin, or analog, fragment, derivative, or variant thereof, operably linked via the heavy chain to DSPAalpha1, or analog, fragment, derivative, or variant thereof.

In another preferred embodiment, the invention relates to a method of preventing arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus and deep vein thrombosis, pulmonary embolism, and acute ischemic stroke, comprising administering to a human in need thereof a therapeutically effective amount of a fusion protein comprising HuSZ51, which binds to P-selectin, or analog, fragment, derivative, or variant thereof, operably linked via the heavy chain to DSPAalpha1, or analog, fragment, derivative, or variant thereof. In a preferred embodiment the fusion protein is administered to a patient suffering acute ischemic stroke more than 3 hours after stroke onset.

In another preferred embodiment, the invention relates to a fusion protein comprising an antibody that binds to activated platelets, or analog, fragment, derivative, or variant thereof, operably linked to a plasminogen activator, or analog, fragment, derivative, or variant thereof, selected from the group consisting of DSPAalpha1, DSPAalpha2, DSPAbeta, and DSPAgamma.

In another preferred embodiment, the invention relates to a fusion protein comprising an antibody that binds to P-selectin, or analog, fragment, derivative, or variant thereof, operably linked to a plasminogen activator, or analog, fragment, derivative, or variant thereof, selected from the group consisting of DSPAalpha1, DSPAalpha2, DSPAbeta, and DSPAgamma.

In another preferred embodiment, the invention relates to a fusion protein comprising an antibody that binds to P-selectin, or analog, fragment, derivative, or variant thereof, operably linked to DSPAalpha2, or analog, fragment, derivative, or variant thereof.

In another preferred embodiment, the invention relates to a fusion protein comprising an antibody that binds to P-selectin, or analog, fragment, derivative, or variant thereof, operably linked to DSPAbeta, or analog, fragment, derivative, or variant thereof.

In another preferred embodiment, the invention relates to a fusion protein comprising an antibody that binds to P-selectin, or analog, fragment, derivative, or variant thereof, operably linked to DSPAgamma, or analog, fragment, derivative, or variant thereof.

In another embodiment, the invention relates to a fusion protein comprising an antibody that binds to P-selectin selected from the group consisting of a monoclonal antibody, a chimeric monoclonal antibody, a humanized monoclonal antibody, a human monoclonal antibody, a Fab dimer antibody, a Fab monomer antibody, an IgG antibody, an analog of IgG antibody, and a single chain antibody, or analog, fragment, derivative, or variant thereof, operably linked to a plasminogen activator selected from the group consisting of DSPAalpha1, DSPAalpha2, DSPAbeta and DSPAgamma, or analog, fragment, derivative, or variant thereof.

In another embodiment, the invention relates to a fusion protein comprising an antibody that binds to P-selectin, or analog, fragment, derivative, or variant thereof, operably linked to a plasminogen activator selected from the group consisting of DSPAalpha1, DSPAalpha2, DSPAbeta and DSPAgamma, or analog, fragment, derivative, or variant thereof, wherein said fusion protein is modified by the addition of other molecules, including, but not limited to polyethylene glycol, biotin, and chondroitin sulfate.

In another embodiment, the invention relates to a fusion protein comprising an antibody that binds to P-selectin, or analog, fragment, derivative, or variant thereof, operably linked to a plasminogen activator selected from the group consisting of DSPAalpha1, DSPAalpha2, DSPAbeta and DSPAgamma, or analog, fragment, derivative, or variant thereof, wherein said antibody does not compete with P-selectin-glycoprotein-ligand-1 (PSGL-1) for binding to P-selectin, compete with the glycoprotein Ib-IX-V complex for binding to P-selectin, or inhibit the adherence of leukocytes to activated platelets.

### Preparation of the Fusion Proteins of the Invention

The fusion proteins according to the preferred embodiment of the invention were generated using standard recombinant DNA, mammalian gene expression, and protein purification technologies. FIG. 1A depicts an example of one such fusion protein, HuSZ51-DSPAalpha1, which is comprised of two light chains consisting of the V_{L} region of an anti-P-selectin antibody fused to the constant region of human Igkappa, and two heavy chains consisting of the V_{H} region of an anti-P-selectin antibody fused to the constant regions of human IgGgamma1 and the mature, secreted form of vampire bat plasminogen activator DSPAalpha1. The construction of anti-P-selectin-DSPAalpha1 fusion protein expression plasmids, and the expression and generation of the anti-P-selectin-DSPAalpha1 fusion protein, are described above.

The mouse monoclonal antibody SZ51, which specifically binds to P-selectin, was obtained from the Jiangsu Institute of Hematology. The DNA sequences of the variable regions of the IgGgamma1 heavy and Igkappa light chain genes of SZ51 were deposited into GenBank (accession numbers gi:4099282 and gi:4099284, respectively). As described in detail above and depicted in FIG. 1B, the cDNA encoding the V_{H} region of SZ51 was cloned into an expression plasmid 5′ to the cDNA encoding the constant region of human Igkappa, and the cDNA encoding the V_{H} region of SZ51 was cloned into an expression plasmid 5′ to the genomic DNA encoding the constant region of human IgGgamma1. The cDNA encoding the mature, secreted form of DSPAalpha1 was inserted 3′ to the CH3 domain of IgGgamma1.

These expression plasmids were subjected to DNA sequencing to confirm the above constructs and the amino acid sequences of the fusion proteins. The amino acid sequences of the chimeric SZ51-V_{L}-human Igkappa light chain (SEQ ID. NO:1) and the chimeric SZ51-V_{H}-human IgGgamma1-DSPAalpha1 heavy chain (SEQ ID NO:2) are shown in FIGS. 2A and 2B, respectively.

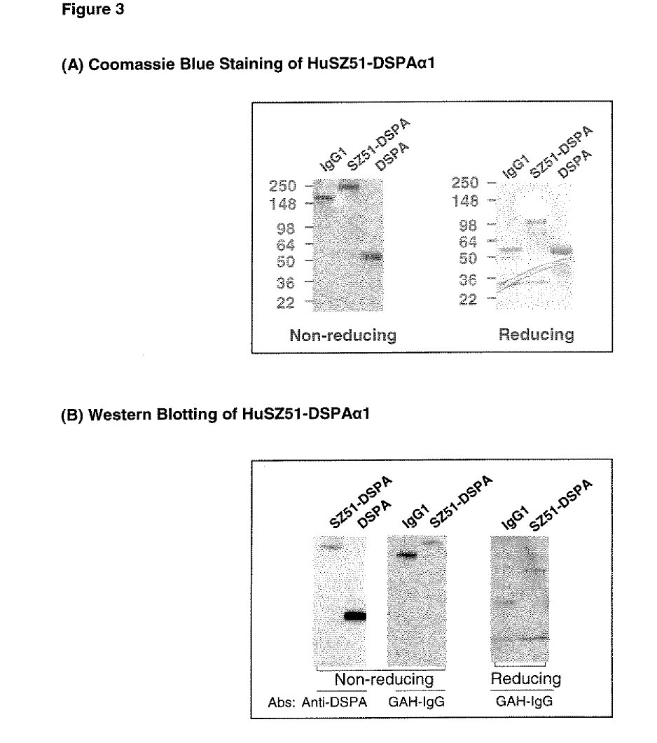

The light and heavy chain expression plasmids were co-transfected into CHO cells and stable transformants expressing anti-P-selectin-DSPA fusion proteins were identified, and then HuSZ51-DSPAalpha1 was purified from the conditioned media of selected positive CHO clones, as described above. HuSZ51-DSPAalpha1 was analyzed by SDS-PAGE and Western blotting, as described in Example 1, the results of which are shown in FIG. 3. As predicted, the purified HuSZ51-DSPAalpha1 fusion protein has an apparent Mr. of ˜250 kDa and is composed of two subunits of ˜108 kDa and ˜23 kDa, corresponding to HuSZ51-V_{H}Cγ₁₋₃-DSPAalpha1 and HuSZ51-V_{L}Cκ, respectively. No significant degradation products or impurities are apparent in the purified HuSZ51-DSPAalpha1 material, as judged by Coomassie blue staining and Western blotting in FIGS. 3A and 3B, respectively.

Using the above methods, as exemplified herein for HuSZ51-DSPAalpha1, fusion proteins of the invention are cloned, expressed, purified, and analyzed for their purity and integrity. Using these methods, a person having ordinary skill in the art would be able to combine different antibody fragments with plasminogen activator fragments to generate the fusion proteins of the invention.

### Testing of the Fusion Proteins of the Invention

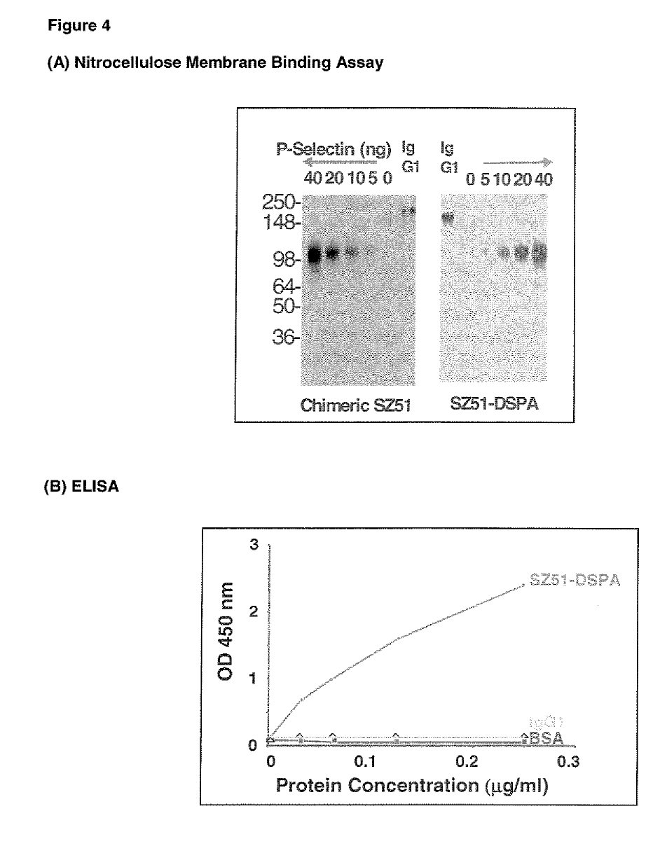

The fusion proteins of the invention, as exemplified by HuSZ51-DSPAalpha1, were generated using standard recombinant DNA, mammalian gene expression, and protein purification technologies as described herein above, and were subsequently analyzed for their purity and integrity as described in Example 1, the results of which are shown in FIG. 3. The fusion proteins of the invention were tested in a variety of in vitro assays to demonstrate utility, namely, that the fusion proteins of the invention retain the ability to bind to activated platelets, degrade fibrin, and induce thrombolysis. These assays are described in detail in Examples 2 to 7 below, the results of which are shown in FIGS. 4 to 9.

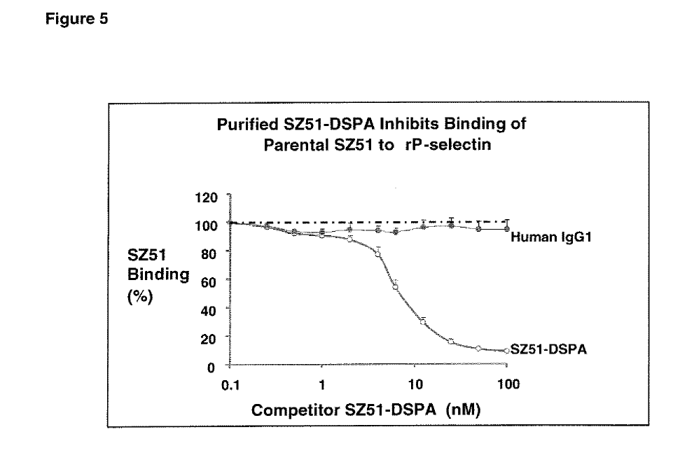

The ability of purified HuSZ51-DSPAalpha1 to bind to P-selectin in vitro was confirmed in three ways: (1) by a nitrocellulose filter binding assay, which measures the ability of HuSZ51-DSPAalpha1 to bind to recombinant P-selectin immobilized on a filter membrane; (2) by ELISA, which measures the ability of HuSZ51-DSPAalpha1 to bind to P-selectin immobilized on plastic; and (3) by competitive ELISA, which measures the ability of HuSZ51-DSPAalpha1 to compete with the parental monoclonal antibody SZ51 for binding to recombinant P-selectin immobilized on plastic. These assays are described in Examples 2 and 3. FIG. 4A shows that HuSZ51-DSPAalpha1 and HuSZ51 were comparable in their ability to bind to P-selectin immobilized on nitrocellulose, and FIG. 4B shows that HuSZ51-DSPAalpha1 bound to P-selectin immobilized on plastic in a dose-dependent fashion. FIG. 5 shows that HuSZ51-DSPAalpha1 competed SZ51 binding to P-selectin in a dose-dependent fashion. These results demonstrated that linking DSPAalpha1 to the C-terminus of the HuSZ51 heavy chain did not adversely affect the ability of the anti-P-selectin antibody to bind to P-selectin in vitro.

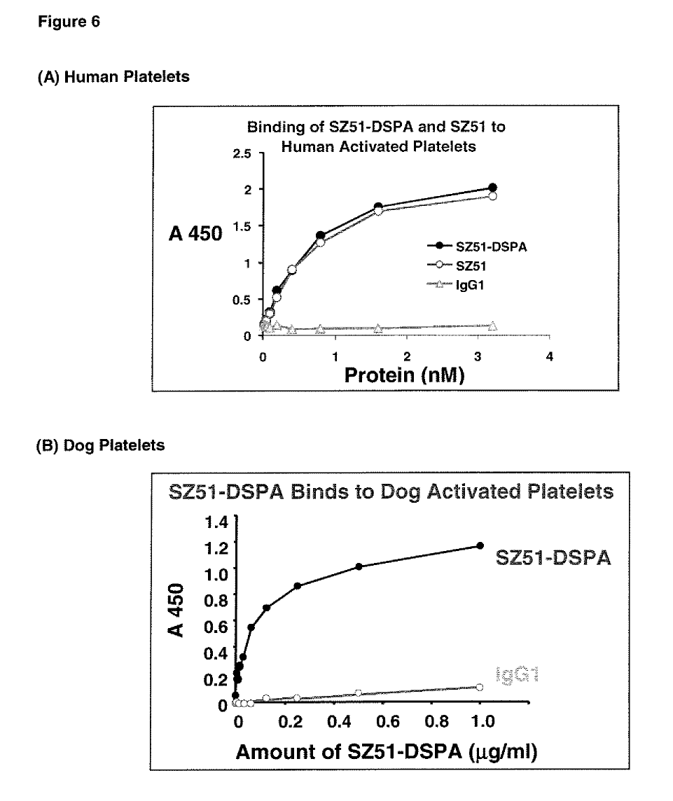

The ability of purified HuSZ51-DSPAalpha1 to bind to activated platelets in vitro was confirmed by ELISA, which measures the ability of HuSZ51-DSPAalpha1 to bind to thrombin-activated platelets coated onto plastic. This assay is described in Example 4 below. FIG. 6A shows that HuSZ51-DSPAalpha1 and SZ51 bound to human thrombin-activated platelets in an indistinguishable manner, and FIG. 6B shows that HuSZ51-DSPAalpha1 bound to dog thrombin-activated platelets in a dose-dependent fashion. These results demonstrated that linking DSPAalpha1 to the C-terminus of the HuSZ51 heavy chain did not adversely affect the ability of the anti-P-selectin antibody to bind to activated platelets in vitro.

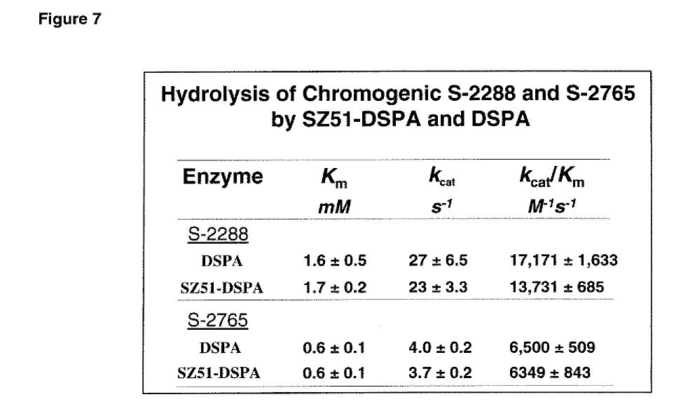

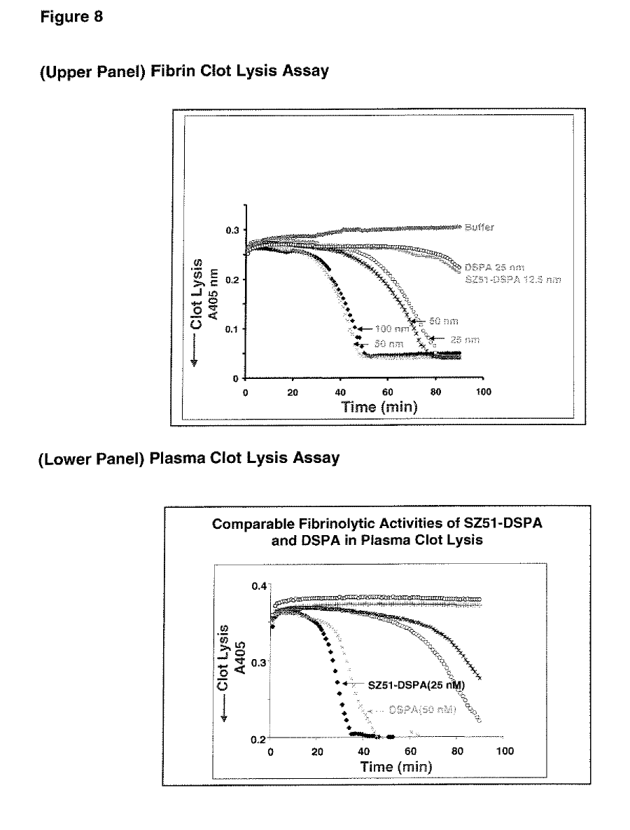

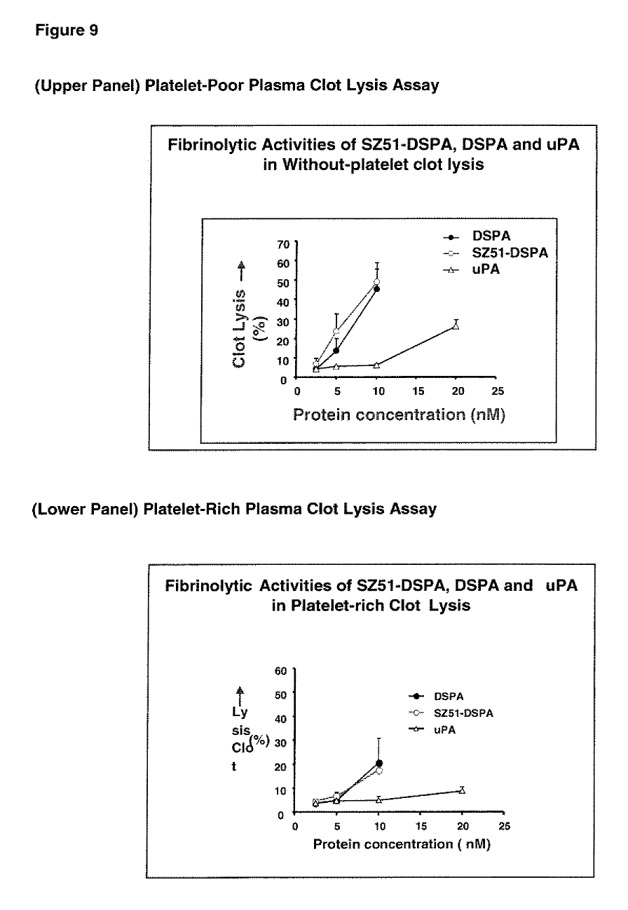

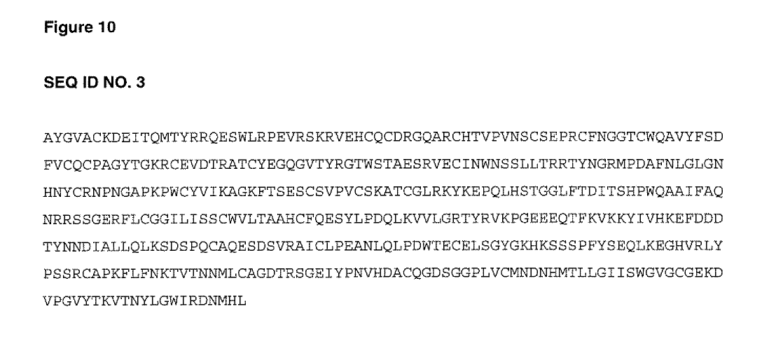

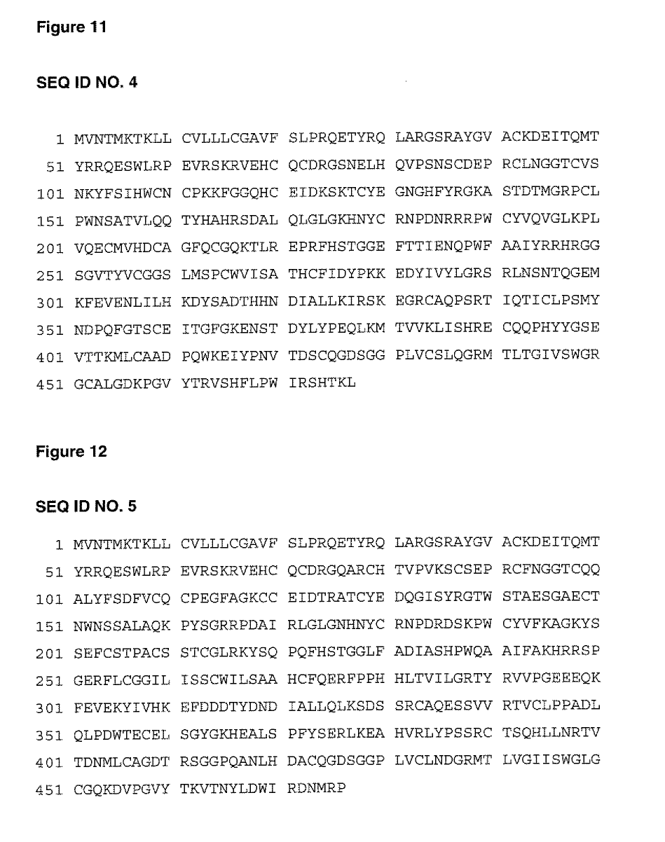

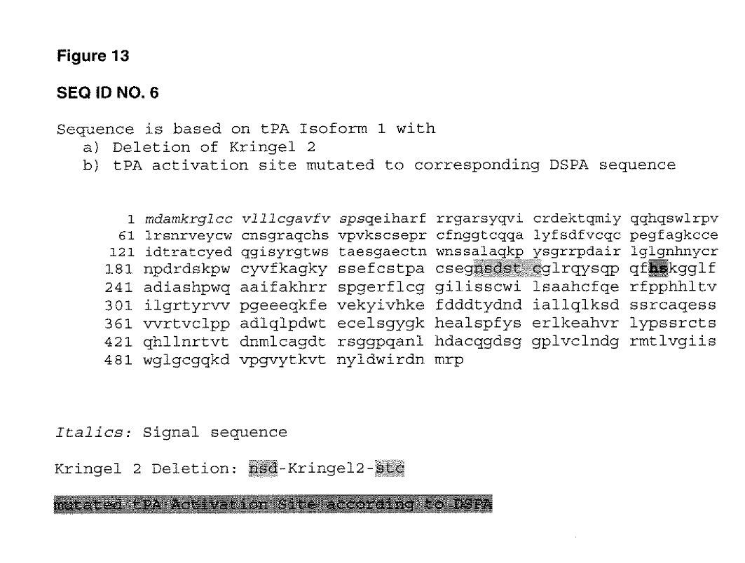

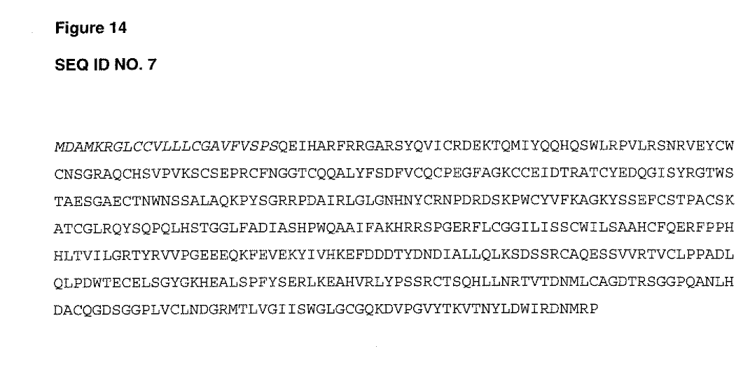

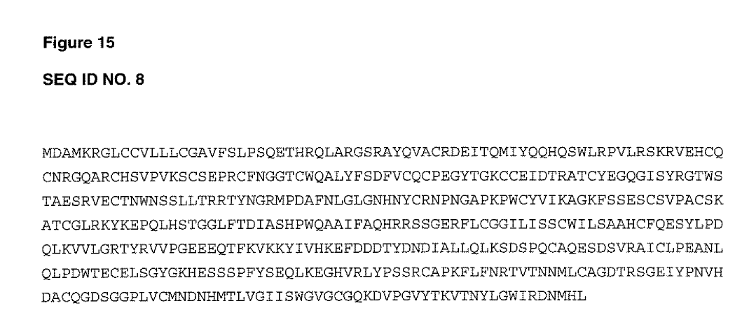

The in vitro catalytic activity of purified HuSZ51-DSPAalpha1 was confirmed in two ways: (1) a chromogenic assay, which measures the ability of HuSZ51-DSPAalpha1 to catalyze hydrolysis of serine protease substrates; and (2) a clot lysis assay, which measures the ability of HuSZ51-DSPAalpha1 to degrade fibrin. These assays are described in Examples 5, 6, and 7. FIG. 7 shows that HuSZ51-DSPAalpha1 and DSPAalpha1 displayed comparable catalytic activity on two serine protease substrates. FIG. 8 (upper panel) shows that, on a molar basis, HuSZ51-DSPAalpha1 and DSPAalpha1 were indistinguishable in their ability to degrade fibrin in the fibrin clot lysis assay, and FIG. 8 (lower panel) shows that HuSZ51-DSPAalpha1 and DSPAalpha1 were comparable in their ability to degrade fibrin in the plasma clot lysis assay. FIG. 9 shows that both HuSZ51-DSPAalpha1 and DSPAalpha1 were superior to uPA in the plasma clot lysis assay, using platelet-poor (upper panel) or platelet-rich (lower panel) plasma. These results demonstrate that linking DSPAalpha1 to the C-terminus of the HuSZ51 heavy chain did not adversely affect the ability of DSPAalpha1 to catalyze hydrolysis of target substrates, and that HuSZ51-DSPAalpha1 was superior to uPA as a thrombolytic agent, in vitro.

The fusion proteins of the invention are tested in an in vivo assay to demonstrate utility, namely, that the fusion proteins of the invention are effective thrombolytic agents, with a lower bleeding risk than the corresponding plasminogen activator alone. This assay is described in Example 8.

The following specific Examples are provided as a guide to assist in the practice of the invention, and are not intended as a limitation on the scope of the invention. It is understood that in the following preparations and examples, combinations of antibody fragments and plasminogen activator fragments are permissible only if such contributions result in stable fusion proteins.

### Example 1

### SDS-PAGE and Western Blot Analysis of an Anti-P-Selectin-DSPA Fusion Protein

The purified HuSZ51-DSPAalpha1 fusion protein was analyzed by SDS-PAGE and by Western blotting. The data indicates that the purified HuSZ51-DSPAalpha1 is intact and free of detectable contaminating material.

1. Analysis of HuSZ51-DSPAalpha1 by SDS-PAGE. Purified HuSZ51-DSPAalpha1, recombinant DSPAalpha1, and human IgG1 were separated on 4 to 10% gradient SDS-PAGE gels under non-reducing and reducing conditions, and stained with Commassie blue. The results are shown in FIG. 3A. Under non-reducing conditions, HuSZ51-DSPAalpha1 has an apparent Mr. of ˜250 kDa, and under reducing conditions, HuSZ51-DSPAalpha1 is composed of two subunits of ˜108 kDa and ˜23 kDa, which correspond to HuSZ51-V_{H}Cγ₁₋₃-DSPAalpha1 and HuSZ51-V_{L}Cκ, respectively. No significant degradation products or impurities are apparent in the purified HuSZ51-DSPAalpha1 material.

2. Analysis of HuSZ51-DSPAalpha1 by Western blotting. Purified HuSZ51-DSPAalpha1, recombinant DSPAalpha1, and human IgG1 were separated on 4 to 10% gradient SDS-PAGE gels under non-reducing and reducing conditions. After the electrophoresis, the proteins on the gels were transferred onto Hybond ECL nitrocellulose membranes (Amersham). The membranes were incubated with the ECL blocking agent (Amersham) for 1 hour at room temperature, followed by three times washing with PBST. The membranes were then incubated with either biotinylated anti-DSPA monoclonal 9B3 (Anti-DSPA, 1:4000) followed by further incubation with HRP-Avidin (1:5000), or with HRP-conjugated goat-anti-human IgG Fc (GAH-IgG, 1:7500). Addition of ECL western blotting detection reagents (Amersham) resulted in chemiluminescent signals captured on standard X-ray film. The results are shown in FIG. 3B. Anti-DSPA detects HuSZ51-DSPAalpha1 as a single species under non-reducing conditions. GAH-IgG detects HuSZ51-DSPAalpha1 as a single species under non-reducing conditions and as two species (HuSZ51-V_{H}Cγ₁₋₃-DSPAalpha1 and HuSZ51-V_{L}Cκ) under reducing conditions. No significant degradation products or impurities are apparent in the purified HuSZ51-DSPAalpha1 material.

Using these methods, other anti-P-selectin-DSPAalpha1 fusion proteins were analyzed for purity and integrity, including HuSZ51Fab′-DSPAalpha1, which lacks the CH2 and CH3 domains in HuSZ51-DSPAalpha1; and scFvSZ51-DSPAalpha1, which is a single chain antibody containing (from N- to C-terminus) the SZ51-V_{L} region followed by the SZ51-V_{H} region and the mature, secreted form of DSPAalpha1. Similar methods are used to analyze other fusion proteins of the invention for purity and integrity.

### Example 2

### Specific P-Selectin Binding Activity of an Anti-P-Selectin-DSPA Fusion Protein

The ability of purified HuSZ51-DSPAalpha1 to bind specifically to P-selectin in vitro was investigated using a nitrocellulose binding assay and by ELISA. The data indicates that HuSZ51-DSPAalpha1 retains the P-selectin binding activity of the original anti-P-selectin monoclonal antibody SZ51.

1. Binding of HuSZ51-DSPAalpha1 to P-selectin on a nitrocellulose membrane. The indicated amounts of recombinant soluble P-selectin (R&D systems), 0, 5, 10, 20, or 40 ng, along with 10 ng of human IgG1, were separated on SDS-PAGE gels and then transferred onto nitrocellulose membranes. One membrane was incubated with HuSZ51, and the second was incubated with HuSZ51-DSPAalpha1. After washing, bound HuSZ51 or HuSZ51-DSPAalpha1 was detected by HRP-conjugated goat anti-human Fc. The intensity of human IgG1 lane, which is also detected by HRP-conjugated goat anti-human Fc was used as a normalization control. FIG. 4A shows that HuSZ51 and HuSZ51-DSPAalpha1 are indistinguishable in their ability to bind to P-selectin immobilized on a nitrocellulose membrane.

2. Binding of HuSZ51-DSPAalpha1 to P-selectin in an ELISA 96-Well plates were coated with 100 μl of recombinant human P-selectin (R&D Systems) per well at a final concentration of 2 μg/ml (reconstituted in PBS), and incubated overnight at 4° C. The coated plates were washed once in washing solution (PBS, pH 7.4 plus 0.05% Tween-20), and then incubated with 200 μl blocking solution (5% milk prepared in PBS) per well for 2 hours at 37° C. After blocking, the plates were washed 3 times with washing solution, and then incubated with 100 μl washing solution containing various amounts of HuSZ51-DSPAalpha1, human IgG1 or BSA for 1 hour at 37° C. The plates were washed 3 times with washing solution, and then incubated with peroxidase-conjugated protein L (Pierce) for 1 hour at 37° C. The plates were washed 4 times with washing solution, and then incubated with 100 μl of TMB/E substrate (ZYMED) for 5 minutes at room temperature. The perioxidase reactions were stopped by the addition of 100 μl 1 N HCl, and the plates were read for absorbance at 450 nm within 5 minutes. FIG. 4B shows that HuSZ51-DSPAalpha1, but not human IgG1, is able to bind to P-selectin immobilized on plastic.

### Example 3

### Competitive Binding of an Anti-P-Selectin-DSPA Fusion Protein to P-Selectin

An ELISA-based competitive binding assay was designed to compare the P-selectin binding affinities of the parental P-selectin antibody, SZ51, and the anti-P-selectin-DSPA fusion protein, HuSZ51-DSPAalpha1. 96-Well plates were coated with 100 μl of recombinant human P-selectin (R & D Systems) per well at a final concentration of 2 μg/ml, and incubated overnight at 4° C. The coated plates were washed once with washing solution (PBS, pH 7.4 plus 0.05% Tween-20), and then incubated with 200 μl blocking solution (5% milk prepared in PBS) per well at 37° C. for 2 hours.

Fifty μl of serially diluted competitors (HuSZ51-DSPAalpha1, or human IgG1 as a negative control) at a final concentration ranging from 0 to 200 nM was added to individual wells, followed by the addition of 50 μl SZ51 (1.6 nM final) to each well, except for the blanks, and then the plates were incubated for 1 hour at 37° C. The plates were washed 3 times with washing solution, and then incubated with a secondary antibody, peroxidase-conjugated anti-mouse IgG (SANTA CRUZ, #S-2031) for 1 hour at 37° C. The plates were washed 4 times with washing solution, and then incubated with 100 μl of TMB/E substrate (ZYMED) for 5 minutes at room temperature. The perioxidase reactions were stopped by the addition of 100 μl 1 N HCl, and the plates were read for absorbance at 450 nm within 5 minutes. The results, shown in FIG. 5, indicated that HuSZ51-DSPAalpha1 was able to inhibit binding of SZ51 to P-selectin in a dose-dependent manner, demonstrating that the anti-P-selectin-DSPA fusion protein retains the same or similar P-selectin binding activity as the anti-P-selectin mouse monoclonal antibody.

### Example 4

### Activated-Platelet Binding Activity of an Anti-P-Selectin-DSPA Fusion Protein

The ability of purified HuSZ51-DSPAalpha1 to bind specifically to human or dog thrombin-activated platelets in vitro was investigated by ELISA. 96-Well plates were coated with a 100 μl solution containing 1×10⁶ thrombin-activated human or dog platelets per well and incubated overnight at 4° C. The coated plates were washed once with washing solution (PBS, pH 7.4 plus 0.05% Tween-20), and then incubated with 200 μl blocking solution (5% milk prepared in PBS) per well at 37° C. for 2 hours. The blocked plates were washed 3 times with washing solution, and then incubated with 100 μl washing solution containing various amounts of HuSZ51-DSPAalpha1, SZ51, or human IgG1 for 1 hour at 37° C. The plates were washed 3 times with washing solution, and then incubated with peroxidase-conjugated protein L (Pierce) for 1 hour at 37° C. The plates were washed 4 times with washing solution, and then incubated with 100 μl of TMB/E substrate (ZYMED) for 5 minutes at room temperature. The perioxidase reactions were stopped by the addition of 100 μl 1 N HCl, and the plates were read for absorbance at 450 nm within 5 minutes. FIG. 6A shows that HuSZ51-DSPAalpha1 and SZ51 are indistinguishable in their ability to bind specifically to human thrombin-activated platelets in vitro. FIG. 6B shows that HuSZ51-DSPAalpha1 is able to bind specifically to dog thrombin-activated platelets in vitro.

### Example 5

### Catalytic Activity of an Anti-P-Selectin-DSPA Fusion Protein

A chromogenic assay (Bringmann, P. et al. (1995), supra) was used to compare the catalytic activity of the anti-P-selectin-DSPA fusion protein, HuSZ51-DSPAalpha1, with that of recombinant DSPAalpha1. S-2288 (D-Ile-Pro-Arg-p-Nitroaniline dihydrochloride) is a chromogenic substrate for a large range of serine proteases. S-2765 (alpha-Benzyloxycarbonyl-D-Arg-Gly-Arg-p-Nitroaniline dihydrochloride) is a chromogenic substrate for factor Xa. Plasminogen activators like DSPAalpha1 cleave the p-Nitroaniline (“pNA”) group from these chromogenic substrates. Reaction mixtures are prepared in 96-well plates in a volume of 150 μl per well containing 10 nM HuSZ51-DSPAalpha1 or 20 nM DSPAalpha1 (an equimolar amount since each HuSZ51-DSPAalpha1 contains two DSPAalpha1 molecules), and 0.1 to 0.8 mM S-2288 or S-2765, in carbonate buffer (0.05 M Na₂CO₃ and 0.1% Tween 80), pH 9.5. The hydrolysis reaction was allowed to proceed at room temperature, and the reaction rate was measured by the change of absorbance value at 405 nm using a microtiter plate reader. The resultant values were converted to [pNA] using standard curves, and the data was plotted against the S-2288 or S-2765 concentrations. The kinetic parameters Kₘ, k_{cat} and Kₘ/k_{cat} were calculated according to the Michaelis-Menten equation with a computerized program (KaleidaGraph 3.0). The results, shown in FIG. 7, demonstrated that HuSZ51-DSPAalpha1 and DSPAalpha1 had similar catalytic activities in vitro.

### Example 6

### Fibrinolytic Activity of an Anti-P-Selectin-DSPA Fusion Protein

Plasminogen activators such as DSPAalpha1 convert plasminogen to plasmin, which in turn degrades fibrin. The rate of fibrin degradation can be monitored by absorbance value at 405 nm (the degradation proceeds with a decrease in the absorbance value). Fibrinolytic activity in vitro was measured in two clot lysis assay formats: (A) using plasminogen and fibrinogen as enzyme and substrate, respectively; and (B) using plasma as source of both enzyme and substrate. (A) In the fibrin clot lysis assay, reaction mixtures were prepared in 96-well plates as follows: 20 μl HuSZ51-DSPAalpha1 (12.5, 25, and 50 nM) or equimolar amounts of DSPAalpha1, 10 μl fibrinogen (20 mg/ml), 1 μl plasminogen (1 U/ml), 2 μl 1 M CaCl₂, 60 μl 0.04 M HEPES, pH 7.0, 0.15 M NaCl, and 0.01% Tween 80 (v/v) were mixed. The mixture was immediately added to another well containing 4 μl thrombin (75 U/ml). The total volume of the mixture was made up to 120 μl with water. After mixing, the absorbance at 405 nm was monitored at 37° C. every minute for 60 minutes. The data was analyzed by KaleidaGraph 3.0. (B) The plasma clot lysis assay was prepared as in (A), except 30 μl reconstituted human plasma (51 mg/ml, Sigma) was used instead of fibrinogen and plasminogen. As shown FIG. 8, the in vitro fibrinolytic activities of HuSZ51-DSPAalpha1 and DSPAalpha1 were indistinguishable in the fibrin clot lysis assay (upper panel) and were comparable in the plasma clot lysis assay (lower panel).

### Example 7

### Thrombolytic Activity of an Anti-P-Selectin-DSPA Fusion Protein In Vitro

The thrombolytic activity of HuSZ51-DSPAalpha1 was assessed in clot lysis assays with platelet-poor (upper panel) or platelet-rich (lower panel) plasma. Assays were performed basically as described in Example 6. Platelet-poor plasma contains approximately 2×10⁴ platelets/μl and platelet-rich plasma contains approximately 2×10⁵ platelets/μl. The fibrinolytic activities of HuSZ51-DSPAalpha1, DSPAalpha1, and uPA (Sigma) were compared in the plasma clot lysis assay, using either platelet-poor (upper panel) or platelet-rich (lower panel) plasma. HuSZ51-DSPAalpha1, DSPAalpha1, or uPA was mixed with I¹²⁵-labeled clots in 1 ml of reconstituted human plasma for 3 hours, and 100 μl of the supernatant was taken to measure soluble radioactivity. Percent lysis is: soluble radioactivity/total radioactivity×100%. The in vitro thrombolytic activity of HuSZ51-DSPAalpha1 was comparable to DSPAalpha1 and was superior to uPA, using either platelet-poor or platelet-rich plasma.

### Example 8

### Thrombolytic Activity of an Anti-P-Selectin-DSPA Fusion Protein In Vivo Dog Femoral Artery Thrombolysis Model

Male Beagle dogs (8-12 kg) are anesthetized with isoflurane (2-3%) and intubated with a tracheal tube. The right and left femoral arteries are isolated and a Transonic flow probe (2.5SB) is placed around the artery. Catheters, filled with saline are inserted into the left and right jugular veins for blood sampling and for administration of HuSZ51-DSPAalpha1, DSPAalpha1, or vehicle. For measurement of hemodynamic parameters, a Miller pressure transducer catheter (2 Fr.) is inserted into the right brachial artery. The incisions are closed with wound clips.

Blood pressure, heart rate and femoral artery flow are monitored throughout the experiment and processed by NOTOCORD data acquisition system. Breathing patterns are monitored visually. Parameters of efficacy, such as time to occlusion after FeCl₂ application, time to reperfusion after drug administration, and duration of reperfusion are obtained from the femoral blood flow measurement. For animals whose vessels do not recanalize following drug administration, reperfusion time is recorded as 240 minutes (the entire observation period) and duration of reperfusion as 0 minutes. Total blood flow perfused into the femoral vascular bed following drug administration is determined by calculating the area under the curve (AUC), and is expressed as a percentage of baseline values. For the prevention vessel, time to occlusion, total duration of reperfusion and area under the curve are calculated as measures of efficacy.

Each of the four nails of the left forepaw is assigned to a bleeding time determination. Bleeding is induced by clipping the cuticle (˜3 mm from the extremity) of the nail using a nail trimmer. The time for the cut to cease bleeding is recorded as primary bleeding time. Re-bleeding time is monitored and recorded for those nails that had previously clotted.

Ex-vivo alpha₂-antiplasmin and DSPA activities are determined by calorimetric assays. Endogenous alpha₂-antiplasmin activity is determined using D-Val-Leu-Lys-p-Nitroanilide as the substrate, and the activity kinetics is measured at 405 nm.

After stabilization of hemodynamic parameters, a blood (4 ml) sample is taken and basal bleeding time is measured. A filter paper (5×6 mm) soaked in a 10% FeCl₂ is applied on the isolated femoral artery for 10 minutes to chemically injure the endothelium locally. This causes the gradual formation of a thrombotic occlusion inside the vessel, and completely occludes the femoral artery (blood flow=0). A period of 40 minutes is allowed for maturation of the thrombus before starting DSPA administration. A second blood sample is collected 25 min after occlusion. Starting 40 minutes after occlusion, DSPAalpha1 (0.1, 0.3 and 1.0 mg/kg), HuSZ51-DSPAalpha1 (0.3 and 0.75 mg/kg) or vehicle (1-2 ml/kg) is injected as a bolus into the jugular vein. Concomitant with drug infusion, FeCl₂ is applied to the second femoral artery to evaluate the effect of HuSZ51-DSPAalpha1 or DSPAalpha1 in preventing thrombus formation. The bleeding time determinations are made and blood samples are collected for ex-vivo alpha₂-antiplasmin and DSPA activities at the following time points: baseline, 5, 30, 60, 120, 180 and 240 minutes after drug administration.

Using this assay, the fusion proteins of the invention display superior thrombolytic activity as compared to DSPAalpha1, with lower bleeding risk.

### Example 9

This example illustrates the preparation of representative pharmaceutical compositions for oral administration containing a compound of the invention:

| A. Ingredients | % wt./wt. |
|---|---|
| Fusion protein of the invention | 20.0% |
| Lactose | 79.5% |
| Magnesium stearate | 0.5% |

The above ingredients are mixed and dispensed into hard-shell gelatin capsules containing 100 mg each, one capsule would approximate a total daily dosage.

| B. Ingredients | % wt./wt. |
|---|---|
| Fusion protein of the invention | 20.0% |
| Magnesium stearate | 0.9% |
| Starch | 8.6% |
| Lactose | 69.6% |
| PVP (polyvinylpyrrolidine) | 0.9% |

The above ingredients with the exception of the magnesium stearate are combined and granulated using water as a granulating liquid. The formulation is then dried, mixed with the magnesium stearate and formed into tablets with an appropriate tableting machine.

| C. Ingredients | | |
|---|---|---|
| Fusion protein of the invention | 0.1 | g |
| Propylene glycol | 20.0 | g |
| Polyethylene glycol 400 | 20.0 | g |
| Polysorbate 80 | 1.0 | g |
| Water | q.s. 100 | mL |

The fusion protein of the invention is dissolved in propylene glycol, polyethylene glycol 400 and polysorbate 80. A sufficient quantity of water is then added with stirring to provide 100 mL of the solution which is filtered and bottled.

| D. Ingredients | % wt./wt. |
|---|---|
| Fusion protein of the invention | 20.0% |
| Peanut Oil | 78.0% |
| Span 60 | 2.0% |

The above ingredients are melted, mixed and filled into soft elastic capsules.

| E. Ingredients | % wt./wt. |
|---|---|
| Fusion protein of the invention | 1.0% |
| Methyl or carboxymethyl cellulose | 2.0% |
| 0.9% saline | q.s. 100 mL |

The fusion protein of the invention is dissolved in the cellulose/saline solution, filtered and bottled for use.

### Example 10

This example illustrates the preparation of a representative pharmaceutical formulation for parenteral administration containing a fusion protein of the invention:

| Ingredients | | |
|---|---|---|
| Fusion protein of the invention | 0.02 | g |
| Propylene glycol | 20.0 | g |
| Polyethylene glycol 400 | 20.0 | g |
| Polysorbate 80 | 1.0 | g |
| 0.9% Saline solution | q.s. 100 | mL |

The fusion protein of the invention is dissolved in propylene glycol, polyethylene glycol 400 and polysorbate 80. A sufficient quantity of 0.9% saline solution is then added with stirring to provide 100 mL of the I.V. solution which is filtered through a 0.2 m membrane filter and packaged under sterile conditions.

### Example 11

This example illustrates the preparation of a representative pharmaceutical composition in suppository form containing a fusion protein of the invention:

| Ingredients | % wt./wt. |
|---|---|
| Fusion protein of the invention | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### Example 12

This example illustrates the preparation of a representative pharmaceutical formulation for insufflation containing a fusion protein of the invention:

| Ingredients | % wt./wt. |
|---|---|
| Micronized fusion protein of the invention | 1.0% |
| Micronized lactose | 99.0% |

The ingredients are milled, mixed, and packaged in an insufflator equipped with a dosing pump.

### Example 13

This example illustrates the preparation of a representative pharmaceutical formulation in nebulized form containing a fusion protein of the invention:

| Ingredients | % wt./wt. |
|---|---|
| Fusion protein of the invention | 0.005% |
| Water | 89.995% |
| Ethanol | 10.000% |

The fusion protein of the invention is dissolved in ethanol and blended with water. The formulation is then packaged in a nebulizer equipped with a dosing pump.

### Example 14

This example illustrates the preparation of a representative pharmaceutical formulation in aerosol form containing a fusion protein of the invention:

| Ingredients | % wt./wt. |
|---|---|
| Fusion protein of the invention | 0.10% |
| Propellant 11/12 | 98.90% |
| Oleic acid | 1.00% |

The fusion protein of the invention is dispersed in oleic acid and the propellants. The resulting mixture is then poured into an aerosol container fitted with a metering valve.

All publications and patents mentioned in the above specification are herein incorporated by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A thrombolytic fusion protein, comprising a targeting protein, which binds to the surfaces of platelets or endothelial cells, whereas said platelets or endothelial cells are activated during a thrombogenic response, said targeting protein is operably linked to DSPA or at least a DSPA domain, whereas the capacity of the DSPA or DSPA domain to activate plasmin is enhanced in the presence of fibrin by more than 650 folds compared to native t-PA.

2. The fusion protein of claim 1, wherein said plasminogen activator is DSPAalpha1.

3. The fusion protein of claim 1, wherein said plasminogen activator is selected from the group consisting of DSPAalpha2, DSPAbeta, and DSPA gamma.

4. The fusion protein according to claim 1, wherein said targeting protein binds to P-selectin.

5. The fusion protein according to claim 1, wherein said targeting protein binds to CD40L, CD63, glycoprotein 1ba or protein disulfide isomerase.

6. The fusion protein according to claim 1, wherein said targeting protein is an antibody.

7. The fusion protein of claim 6, wherein said antibody does not compete with P-selectin-glycoprotein-ligand-1 for binding to P-selectin.

8. The fusion protein of claim 6, wherein said antibody does not compete with glycoprotein 1b-IX-V for binding to P-selectin.

9. The fusion protein of claim 6, wherein said antibody does not inhibit the adherence of leukocytes to activated platelets.

10. The fusion protein of claim 6 wherein said antibody is a monoclonal antibody.

11. The fusion protein of claim 10, wherein said monoclonal antibody is a single chain antibody, a Fab dimer antibody, or an IgG antibody.

12. The fusion protein of claim 1 including a protein compromising an amino acid sequence selected from the group of SEQ ID NO. 3 to SEQ ID NO. 8 or any protein with an amino acid sequence with at least 70% identity thereto with essentially the same biological activity.

13. A pharmaceutical composition, comprising a fusion protein according to claim 1, which composition comprises a pharmaceutically acceptable excipient and a therapeutically effective amount of said fusion protein.

14. A method for inducing thrombolysis, comprising administering a therapeutically effective amount of a fusion protein according to claim 1 to a patient in need thereof.

15. The method of claim 14, wherein said method is to treat arterial thrombosis, acute coronary syndromes, including ST-elevated myocardial infarction, non-ST-elevated myocardial infarction and unstable angina, catheter-induced thrombosis, dissolution of ventricular mural thrombus, left atrial thrombus or prosthetic valve thrombus and deep vein thrombosis, pulmonary embolism, or acute ischemic stroke.

16. The method of claim 15, wherein the fusion protein is administered to a patient suffering acute ischemic stroke more that 3 hours after stroke onset.

17. A kit, comprising the fusion protein according to claim 1.

18. A kit, comprising DNA sequences encoding the fusion protein components according to claim 1.

19. A gene therapy composition, comprising the DNA encoding the fusion protein consisting of the amino acid sequences of SEQ ID NO. 1 and SEQ ID NO. 2, in combination with a therapeutically effective amount of a gene therapy vector.

20. A thrombolytic fusion protein, comprising a targeting protein that binds to P-selectin, wherein said targeting protein is a chimeric mouse-human monoclonal antibody, which is operably linked to DSPAalpha1, or analog, fragment, derivative, or variant thereof.

21. The fusion protein of claim 20, wherein said chimeric mouse-human monoclonal antibody is HuSZ51.

22. The fusion protein of claim 21, wherein said fusion protein comprises the amino acid sequences of SEQ ID NO. 1 and SEQ ID NO. 2.


SEQUENCE LISTING (8 sequences)

SEQ ID NO: 1 - protein, 235 aa, artificial
  HuSZ51-VLCkappa Light Chain
MGWSCIILFLVATATGVHSELQMTQSPSSLSASVGDRVTISCSASQDISNYLNWYQQKPDGPVKLLIYYTTSNLHSGVPSRFSGSGSGTDYSLTISNLEPEDIATYYCQQYNKVPWTFGGGTKLEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 908 aa, artificial
  HuSZ51-VHCkappa1-3-DSPAalpha1 heavy chain
MGWSCIILFLVATATGVHSQVQLLESGPELKKPGETVKISCKASGYTFTNYGMNWVKQAPGKGLKWMGWINTYTGEPIYADDFKGRFAFSLETSASTAHLQINSLKNEDMATYFCARGRGGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAYGVACKDEITQMTYRRQESWLRPEVRSKRVEHCQCDRGQARCHTVPVNSCSEPRCFNGGTCWQAVYFSDFVCQCPAGYTGKRCEVDTRATCYEGQGVTYRGTWSTAESRVECINWNSSLLTRRTYNGRMPDAFNLGLGNHNYCRNPNGAPKPWCYVIKAGKFTSESCSVPVCSKATCGLRKYKEPQLHSTGGLFTDITSHPWQAAIFAQNRRSSGERFLCGGILISSCWVLTAAHCFQESYLPDQLKVVLGRTYRVKPGEEEQTFKVKKYIVHKEFDDDTYNNDIALLQLKSDSPQCAQESDSVRAICLPEANLQLPDWTECELSGYGKHKSSSPFYSEQLKEGHVRLYPSSRCAPKFLFNKTVTNNMLCAGDTRSGEIYPNVHDACQGDSGGPLVCMNDNHMTLLGIISWGVGCGEKDVPGVYTKVTNYLGWIRDNMHL

SEQ ID NO: 3 - protein, 441 aa, artificial
  plasminogen activator
AYGVACKDEITQMTYRRQESWLRPEVRSKRVEHCQCDRGQARCHTVPVNSCSEPRCFNGGTCWQAVYFSDFVCQCPAGYTGKRCEVDTRATCYEGQGVTYRGTWSTAESRVECINWNSSLLTRRTYNGRMPDAFNLGLGNHNYCRNPNGAPKPWCYVIKAGKFTSESCSVPVCSKATCGLRKYKEPQLHSTGGLFTDITSHPWQAAIFAQNRRSSGERFLCGGILISSCWVLTAAHCFQESYLPDQLKVVLGRTYRVKPGEEEQTFKVKKYIVHKEFDDDTYNNDIALLQLKSDSPQCAQESDSVRAICLPEANLQLPDWTECELSGYGKHKSSSPFYSEQLKEGHVRLYPSSRCAPKFLFNKTVTNNMLCAGDTRSGEIYPNVHDACQGDSGGPLVCMNDNHMTLLGIISWGVGCGEKDVPGVYTKVTNYLGWIRDNMHL

SEQ ID NO: 4 - protein, 477 aa, artificial
  plasminogen activator
MVNTMKTKLLCVLLLCGAVFSLPRQETYRQLARGSRAYGVACKDEITQMTYRRQESWLRPEVRSKRVEHCQCDRGSNELHQVPSNSCDEPRCLNGGTCVSNKYFSIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCAGFQCGQKTLREPRFHSTGGEFTTIENQPWFAAIYRRHRGGSGVTYVCGGSLMSPCWVISATHCFIDYPKKEDYIVYLGRSRLNSNTQGEMKFEVENLILHKDYSADTHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKEIYPNVTDSCQGDSGGPLVCSLQGRMTLTGIVSWGRGCALGDKPGVYTRVSHFLPWIRSHTKL

SEQ ID NO: 5 - protein, 476 aa, artificial
  plasminogen activator
MVNTMKTKLLCVLLLCGAVFSLPRQETYRQLARGSRAYGVACKDEITQMTYRRQESWLRPEVRSKRVEHCQCDRGQARCHTVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSSTCGLRKYSQPQFHSTGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 6 - protein, 480 aa, artificial
  plasminogen activator
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDSTCGLRQYSQPQFHSKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 7 - protein, 476 aa, artificial
  plasminogen activator
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSKATCGLRQYSQPQLHSTGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 8 - protein, 476 aa, artificial
  plasminogen activator
MDAMKRGLCCVLLLCGAVFSLPSQETHRQLARGSRAYQVACRDEITQMIYQQHQSWLRPVLRSKRVEHCQCNRGQARCHSVPVKSCSEPRCFNGGTCWQALYFSDFVCQCPEGYTGKCCEIDTRATCYEGQGISYRGTWSTAESRVECTNWNSSLLTRRTYNGRMPDAFNLGLGNHNYCRNPNGAPKPWCYVIKAGKFSSESCSVPACSKATCGLRKYKEPQLHSTGGLFTDIASHPWQAAIFAQHRRSSGERFLCGGILISSCWILSAAHCFQESYLPDQLKVVLGRTYRVVPGEEEQTFKVKKYIVHKEFDDDTYDNDIALLQLKSDSPQCAQESDSVRAICLPEANLQLPDWTECELSGYGKHESSSPFYSEQLKEGHVRLYPSSRCAPKFLFNRTVTNNMLCAGDTRSGEIYPNVHDACQGDSGGPLVCMNDNHMTLVGIISWGVGCGQKDVPGVYTKVTNYLGWIRDNMHL